(19) United States
(12) Patent Application Publication
(10) Pub. No.: US 2023/0029485 A1
(43) Pub. Date: Feb. 2, 2023

(54) METHODS FOR COMPUTATIONAL IDENTIFICATION OF FUNCTIONAL CHANGES TO AMINO ACID RESIDUES CAUSED BY GENETIC VARIANTS, AND METHODS OF COMPUTATIONAL PREDICTION OF CHEMOTHERAPY EFFICACY USING MACHINE LEARNING APPLIED TO GENE EXPRESSION DATA

(71) Applicant: George Mason University, Fairfax, VA (US)

(72) Inventors: Mohsin Saleet JAFRI, Potomac, MD (US); Soukaina AMNIOUEL, Fairfax, VA (US); John Russell HAMRE, III, Monrovia, MD (US)

(21) Appl. No.: 17/855,840

(22) Filed: Jul. 1, 2022

Related U.S. Application Data
(60) Provisional application No. 63/217,804, filed on Jul. 2, 2021.

(51) Int. Cl. G06N 20/00; G16H 70/60

(52) U.S. Cl. CPC G06N 20/00; G16H 70/60

(57) ABSTRACT

One or more computer-implemented methods of using molecular dynamics simulations combined with machine learning to define the ensemble of genomic structures of variants to predict changes in drug and ligand binding.

### CROSS-REFERENCE TO RELATED APPLICATIONS

The present application claims the benefit of U.S. Provisional Patent Application Ser. No. 63/217,804 (filed on Jul. 2, 2021), the disclosure of which is incorporated herein by reference in its complete entirety.

### TECHNICAL FIELD

The present disclosure relates to one or more machine learning methods useful in the field of drug development and therapy. In particular, the present disclosure sets forth, describes, and illustrates one or more computer-implemented methods for analyzing features extracted from molecular simulations of genomic variants to achieve accurate prediction of drug resistance, drug target amino acid residues, and peptide drug candidates. Such computer-implemented methods may be implemented, in particular, for treatment of cancer, Alzheimer's disease treatment, and anti-viral treatment of SARS-CoV-2.

The present disclosure also relates to machine learning methods using gene expression data of cancer tumor samples to predict drug response by evaluating their training performance for diagnostic utility. In particular, the present disclosure sets forth, describes, and illustrates one or more computer-implemented methods that facilitate the identification of novel gene signatures as reliable determinants of drug response in chemotherapy treatments of cancer.

### BACKGROUND

In the field of cancer treatment, variants have been associated with causing the dysfunction underlying the cancer, conferring resistance to cancer therapy drugs, or both. For example, over-expression of anti-apoptotic genes, and under-expression of pro-apoptotic genes, can result in the lack of cell death (apoptosis) that is characteristic of cancer. Other targets include cell grown genes and cell growth checkpoint genes. Other drugs target progression and spread of the cancer. One of the major causes of failed cancer drug treatment is drug resistant variants. Cancer drug resistance is a major reason that cancer drug therapy fails resulting in loss of life, increased patient suffering and an increase in treatment expense. Cancer drug resistance often arises from the emergence of genetic variants in the target gene, which manifest as phenotypic variants of the protein. Unfortunately, many genetic variants remain unclassified as to their effect on cancer drug treatment because experimental verification is costly and time consuming.

### DRAWINGS

The patent or application file contains at least one drawing executed in color. Copies of this patent or patent application publication with color drawing(s) will be provided by the Office upon request and payment of the necessary fee.

The various advantages of the examples of the present disclosure will become apparent to one skilled in the art by reading the following specification and appended claims, and by referencing the following drawings, in which:

FIG. 1 illustrates a plot of residue versus rank of angles in variants that cause an increased aggregation using a computer-implemented method in accordance with one or more embodiments set forth and described herein.

FIG. 2 illustrates a plot of residue versus rank of angles in variants that cause an increased aggregation using a computer-implemented method in accordance with one or more embodiments set forth and described herein.

FIG. 3 illustrates a plot of residue versus rank for results of ranking data set capable of sorting by variant by disease, using a computer-implemented method in accordance with one or more embodiments set forth and described herein.

FIG. 4 illustrates a plot of residue versus rank for results of ranking entire data set by disease, using a computer-implemented method in accordance with one or more embodiments set forth and described herein.

FIG. 5 illustrates a plot of residue versus rank for results of ranking entire data set by mutation, using a computer-implemented method in accordance with one or more embodiments set forth and described herein.

FIG. 6 illustrates a plot of residue versus rank based on average age of onset, using a computer-implemented method in accordance with one or more embodiments set forth and described herein.

FIGS. 7A and 7B illustrate plots of residue versus rank, using a computer-implemented method in accordance with one or more embodiments set forth and described herein.

FIG. 8 illustrates a confusion matrix generated from the 10 residues to check the accuracy of the models in accordance with one or more embodiments set forth and described herein.

FIG. 9 is a table of the average age of onset along with a mutation.

FIG. 10 is a table of drugs used in clinical trials and the residues they targeted.

FIG. 11 is a table of drugs that failed clinical trials, mechanism class, and results.

FIG. 12 is a table of the ranking of the residues.

FIG. 13 is a list, generated from a classification tree using orange3 data mining software, of the angles that could separate the WT from the non-WT points by >0 percent in order to predict variants of Alzheimer's (FAD, Familial Alzheimer's Disease and CAA, Cerebral Amyloid Angiopathy) from wild-type.

FIG. 14 is a ranking of the identified angles with their information gain generated, using a computer-implemented method in accordance with one or more embodiments set forth and described herein.

FIG. 15 is a ranking of the identified angles with their information gain generated, using a computer-implemented method in accordance with one or more embodiments set forth and described herein.

FIG. 16 is a ranking a data set capable of sorting by variant by disease, with their information gain generated, using a computer-implemented method in accordance with one or more embodiments set forth and described herein.

FIG. 17 is a ranking the entire data set by disease, with their information gain generated, using a computer-implemented method in accordance with one or more embodiments set forth and described herein.

FIG. 18 is a ranking the entire data set by mutation, with their information gain generated, using a computer-implemented method in accordance with one or more embodiments set forth and described herein.

FIG. 19 is a ranking the entire data set by results based on average age of onset, with their information generated, using a computer-implemented method in accordance with one or more embodiments set forth and described herein.

FIG. 20 is a table illustrating the distances calculated from the phi an psi dihedral angles as compared to nsp10/nsp16 data.

FIGS. 21A and 21B illustrate a wild-type (green) and G94D (magenta) overlay, and a wild-type (green) and Y96F (cyan) overlay.

FIGS. 22A, 22B, and 22C illustrate a histidine rotamer in G94D (FIG. 22A), K93E (FIG. 22B), and H80A (FIG. 22C) turning approximately 90 degrees G94D and K93E and turning to 45 degrees in the H80A.

FIG. 23 illustrates variants R78A (blue) and R78G (tv red).

FIGS. 24A through 24G illustrate a comparison of the Y96“X” variants to the interaction regions of nsp10 to nsp14, and to nsp16.

FIGS. 25A and 25B illustrate the top dihedral angles in the peptides with respect to determining accuracy of random forest machine learning applied.

FIG. 26 is a table listing predictions using random forest of variants with known MTase activity in whole protein data designated as low (L), middle (M), or high (H) MTase activity, and new variants derived from other coronavirus sequences and their prediction of a future candidate “C” for testing or not a candidate “N.”

FIG. 27 illustrates a 3D image of PCA space of the original variants with known empirical data.

FIG. 28 is a plot of new PCA space including all new variants PC1 vs PC5.

FIGS. 29A through 29C are 3D simulations derived via the computer-implemented method, in accordance with one or more embodiments.

FIG. 30 is a 3D simulation of PDB ID 600k with the average WT structure that was found in-silico, derived via the computer-implemented method, in accordance with one or more embodiments.

FIGS. 31A through 31C are 3D simulations derived via the computer-implemented method, in accordance with one or more embodiments.

FIG. 32 is a table of the distance (angstroms) between specific residue side chains as compared to Kd or Ki concentrations found from surface plasmon resonance.

FIG. 33 is a table of the average distances (angstroms) measured between the residues, versus the log of the Ki of Venetoclax.

FIG. 34 illustrates an example of the distances measured in Angstroms across the binding channel in the wild-type protein.

FIG. 35 is a classification tree derived via the computer-implemented method, in accordance with one or more embodiments.

FIG. 36 a 3D scatter plot, derived via the computer-implemented method, in accordance with one or more embodiments.

FIG. 37 is a chart of the results of the MDPPM and other predictive software.

FIG. 38 is a table of measured Euclidean distances compared to Kd, Ki, and predicted values.

FIGS. 39A through 39C are 3D simulations, derived via the computer-implemented method, in accordance with one or more embodiments.

FIG. 40 is a 3D simulation illustrating a measured distance (angstroms) between an F104L (white) OD2 atom of residue D111 and a α5 helix.

FIG. 41 is a 3D simulation illustrating E152A (cyan) and a α2 helix.

FIGS. 42 through 53 illustrates computer-implemented methods, in accordance with one or more embodiments set forth and described herein.

### DESCRIPTION

The method herein is directed to the identification and classification of different kinds of targets.

Recent advances in the ability to generate molecular data, as well as parallel advances in the fields of artificial intelligence, specifically machine learning (ML), have led to opportunities to understand these resistance mechanisms and develop treatment strategies to overcome resistance. Due to significant molecular heterogeneity observed across tumors, there are often many different molecular features and feature combinations that can lead a model to predict a particular drug response.

An example of a genetic variation occurring in a cancer target gene is related to BCL-2 (B-cell lymphoma 2), which is encoded in humans by the BCL-2 gene. BCL-2 is a regulator protein that regulates cell death by either inhibiting (anti-apoptotic) or inducing (pro-apoptotic) apoptosis. Damage to the BCL-2 gene has been identified as a cause of numerous cancers, including melanoma, breast, prostate, chronic lymphocytic leukemia, and lung cancer, and as being a possible cause of schizophrenia and autoimmunity. It has been found that it is the genetic variability of BCL-2 which is one of the causes of resistance to cancer treatments. In accordance with one or more embodiments illustrated, set forth, and/or described herein, a computer-implemented method is provided that when executed, identifies BCL-2 genetic drug resistant variants to determine effective optional treatments for patients. One or more embodiments are illustrated, set forth, and/or described herein as relating to computer-implemented methods that when executed, determine genetic drug resistant variants of the BCL-2 gene. This disclosure contemplates, however, that the one or more embodiments can be practiced without departing from the spirit and scope of this disclosure and is not limited solely to the examples set forth herein. It is to be understood that the disclosed innovation is applicable to identification of genetic variants relating to a myriad of other genes.

Venetoclax is a BCL-2 (B-cell lymphoma-2) inhibitor that has been successful in treating many B-cell lymphoproliferative diseases such as chronic lymphocytic leukemia (CLL) and diffuse large B-cell lymphoma. Venetoclax blocks the anti-apoptotic activity of BCL-2 by binding the BH3 binding pocket, preventing binding of the natural ligands and subsequently promoting cell death. Venetoclax resistance, however, is a major problem for patients who acquire variants while undergoing therapy. In one study, CLL patients developed Venetoclax resistance in two out of three cases due to genetic variants of BCL-2.

Colorectal cancer (CRC) is a heterogeneous disease characterized by different genomic landscape representing a therapeutic challenge, which is further complicated by the common occurrence of several molecular alterations that confer resistance to chemotherapy and targeted agents. CRC is the most frequent malignant disease of the gastrointestinal tract, the third most frequent cancer affecting both men and women and is one of the leading causes of cancer-related morbidity and mortality despite widespread, effective measures of preventive screening, and major advances in treatment options. More than half of colorectal adenocarcinomas are still diagnosed only when the disease involves regional or distant structures.

Chemotherapy remains one of the most commonly used treatments for CRC patients, and is usually combined with surgery, radiotherapy, immunotherapy, and targeted molecular therapy. Since the 1950s, 5-fluorouracil (5-FU)-based chemotherapy has constituted the backbone of chemotherapeutic regimens for patients with CRC. 5-FU's active metabolites disrupt the synthesis of both DNA and RNA via a mechanism involving the folate metabolic pathway. Nonetheless, the benefits of 5-FU treatment are often short-lived, and many treated patients do not reach complete eradication of tumor cells, resulting in poor outcomes due to recurrence after 5-FU therapy.

While 5-fluorouracil used as single agent in patients with metastatic colorectal cancer has an objective response rate around 20%, the administration of FOLFIRI (FOL=Leucovorin Calcium (Folinic Acid), F=Fluorouracil and IRI=Irinotecan Hydrochloride) or (FOL=Leucovorin Calcium (Folinic Acid), F=Fluorouracil and OX=Oxaliplatin) results in significantly increased response rates and improved survival. These drugs have been used as initial intensive therapy for metastatic CRC in patients with good tolerance.

Despite advances in cytotoxic therapy, resistance to chemotherapy remains one of the greatest challenges in long-term management of incurable metastatic disease and eventually contributes to death as tumors accumulate means of evading treatment. In these cases, patients are typically given a combination of cytotoxic chemotherapy often in conjunction with a targeted therapy. Drug resistance and the resulting ineffectiveness of the drug treatment are responsible for up to 90% of the cancer related deaths. Resistance to cytotoxic chemotherapy occurs by several mechanisms, such as decreased intracellular drug concentration, altered metabolism, or alterations to targets of the therapy. Some of these concepts translate into mechanisms of resistance to novel targeted therapies, however, often resistance is more complex, at times not involving mutation but molecular pathway alteration, something not seen with evasion of traditional chemotherapy.

Typically, chemotherapy destroys cancer cells mostly through induction of apoptosis by damaging DNA and inhibiting cell cycle progression. Eventually, cancer cells acquire diverse strategies that decrease the efficacy of many therapeutic agents leading to chemoresistance. The underlying mechanisms conferring resistance to therapy have been broadly classified into two forms: intrinsic or acquired resistance. Each of these forms is important in determining initial and subsequent lines of treatment. Acquired resistance occur when changes in the genetic makeup of cells over time result in therapy-resistant cells. Whereas intrinsic or innate resistance can either be soluble factor mediated drug resistance or cell-adhesion mediated drug resistance. Chemokines, growth factors and cytokines are known to induce the soluble factor mediated drug resistance.

Alzheimer's disease, the most common form of dementia, currently has no cure. There are only temporary treatments that reduce symptoms and the progression of the disease. Alzheimer's is characterized by the prevalence of plaques of aggregated amyloid β peptide. In accordance with one or more embodiments illustrated, set forth, and/or described herein, a computer-implemented method is provided that when executed, identifies specific amino acid residues associated with causing the pathology in Alzheimer's disease.

Coronaviruses, including the recent pandemic strain SARS-CoV-2, use a multifunctional 2′-O-methyltransferase (2′-O-MTase) to restrict a host defense mechanism by methylating the viral RNA. The non-structural protein 16 2′-O-MTase (nsp16) becomes active when non-structural protein 10 (nsp10) and nsp16 interact. In accordance with one or more embodiments illustrated, set forth, and/or described herein, a computer-implemented method is provided that when executed, identifies analogous nsp10 peptides that have the ability to bind nsp16 with equal to or higher affinity than those naturally occurring and thus allow the host to remove viral RNA.

An important challenge in biology is the accurate prediction of the connection and causation between the genetic makeup of an organism (genome) and the totality of all phenotypes or observable physiological traits and characteristics, also known as genome to phenome. Genomic variations frequently alter wild-type physiological traits.

In accordance with one or more embodiments illustrated, set forth, and/or described herein, a computer-implemented method is provided that when executed, evaluates predictive biomarkers of FOLFOX and FOLFIRI in CRC patients.

In accordance with one or more embodiments illustrated, set forth, and/or described herein, in reference to Alzheimer's disease, over 38,000 simulated structures, generated from Replica Exchange Molecular Dynamics (REMD) simulations, exploring different conformations of the Aβ42 mutants and wild-type peptides are used to study the relationship between amyloid β torsional angles and disease measures. Machine learning techniques are applied to characterize the data and classify how much they influenced the predicted variant of Alzheimer's Disease. Data mining software facilitate use of these techniques, generation of lists, and ranking of the data. The test and score module coupled with the confusion matrix module analyzed data with calculations of specificity and sensitivity. These methods allowed for developing results in each method of potentially important residues. One or more embodiments utilizes an algorithm that evaluates all the methods to generate a predetermined number (e.g., ten) of residues that are the most important in characterizing aggregation. These conclusions are developed by analyzing the results and evaluating them based on frequency and rank. Since drugs target specific residues, this disclosure may identify new drugs to reduce aggregation of amyloid β.

In accordance with one or more embodiments illustrated, set forth, and/or described herein, this disclosure relates to the finding that nsp10 derived peptides can disrupt viral methyltransferase activity via interaction of nsp16. A new set of peptides are developed herein based on conserved regions of the nsp10 protein throughout the Coronaviridae species and tested these to known methyltrasferase variant empirical values.

In accordance with one or more embodiments illustrated, set forth, and/or described herein, a computer-implemented method is provided that when executed, classifies one or more variants in a patient's cancer. Such identification can impact a treatment protocol and effectiveness of cancer drug therapy. In accordance with one or more embodiments illustrated, set forth, and/or described herein, a computer-implemented method is provided that when executed, predict drug resistance of genes related to cancer.

In accordance with one or more embodiments illustrated, set forth, and/or described herein, a computer-implemented method is provided that when executed, identifies drug resistant variants of genes related to Alzheimer's.

In accordance with one or more embodiments illustrated, set forth, and/or described herein, a computer-implemented method is provided that when executed, identifies drug resistant variants of genes related to SARS-Cov-2. In accordance with one or more embodiments illustrated, set forth, and/or described herein, a computer-implemented method is provided that when executed, identifies drug resistant variants of genes related to various diseases and infections other than cancer, Alzheimer's, and SARS-Cov-2.

In accordance with one or more embodiments illustrated, set forth, and/or described herein, a computer-implemented method is provided that when executed, classifies genetic anomalies associated with a myriad of genetic disorders. In accordance with one or more embodiments illustrated, set forth, and/or described herein, a computer-implemented method is provided that when executed, identifies genetic profiles and determines modifications in genomic profiles as they relate to corresponding phenotypes and physiological traits and characteristics.

In accordance with one or more embodiments illustrated, set forth, and/or described herein, a computer-implemented method is provided that when executed, measure the phi and psi angles within a protein backbone.

In accordance with one or more embodiments illustrated, set forth, and/or described herein, a computer-implemented method is provided that when executed, determines factors that initiate the development of tumors related to CRC. In accordance with one or more embodiments illustrated, set forth, and/or described herein, a computer-implemented method is provided that when executed, determines the responsiveness or resistance of tumors related to CRC to anti-tumor agents.

In accordance with one or more embodiments illustrated, set forth, and/or described herein, a computer-implemented method is provided that when executed, applies a molecular dynamic machine learning predictive methodology of the phi and psi angles of the amino acid backbone of BCL-2 protein to predict drug-resistant BCL-2 proteins. In accordance with such one or more embodiments, the variant structures all show shifts in residue positions that occlude the binding groove, which have been ascertained to be primary contributors to drug resistance.

In accordance with one or more embodiments illustrated, set forth, and/or described herein, accurate information on genetic variants is provided that will enable medical teams, researchers, and other professionals to have a more accurate understanding of a genetic profile of a patient suffering from a disease to provide a more accurate treatment protocol therefor.

In accordance with one or more embodiments illustrated, set forth, and/or described herein, a computer-implemented method is provided that when executed, identifies genetic variants of cancer-related genes. Specific genetic variants are used to obtain suitable predictions of phenotypic or pathogenic changes caused by the identified genetic variants.

In accordance with one or more embodiments illustrated, set forth, and/or described herein, where data and/or information about genetic structure and function is limited, molecular dynamic phenotype predictive modeling (MDPPM) is applied to predict phenotypic changes by applying machine learning to a set of phi and psi angles of an ensemble of protein structures for all genetic variants by using molecular dynamics simulations.

The computer-implemented method in accordance with one or more embodiments illustrated, set forth, and/or described herein, is applicable for a myriad of proteins. In particular, in the case of the BCL-2 gene, the computer-implemented method is to predict drug resistance and the effectiveness of the inhibition caused by each genetic variant.

Execution of MDPPM provides an accurate method of predicting variant phenotypes and disease severity through structural analysis of a protein over time. The model begins by taking three-dimensional protein structures from databanks, simulating the entire structure or portions thereof by using molecular dynamics software, then utilizing machine learning to predict what the variant classification or disease-state phenotype will be. The computer-implemented method in accordance with one or more embodiments illustrated, set forth, and/or described herein utilizes MDPPM, while developing further an investigational study of natural variants of a nsp10-derived peptide and its relationships to methyltransferase activity.

In accordance with one or more embodiments illustrated, set forth, and/or described herein, a computer-implemented method is provided that when executed, predicts whether a genetic variant confers drug resistance or is drug sensitive using the target gene BCL-2 and the anti-cancer drug Venetoclax. The computer-implemented method applies machine learning to features extracted from molecular simulations to achieve a greater than 90% prediction accuracy for one or more initial examples of drug resistance.

As used herein, the term “gene” refers to a sequence of nucleotides in DNA or RNA that encodes the synthesis of either RNA or a protein. As used herein, the term “protein” is used interchangeably with the term “macromolecule,” and refers to a biomolecule that is comprised of one or more long chains of amino acid residues.

Data Mining of Molecular Simulations Suggest Key Amino Acid Residues for Aggregation, Signaling, and Drug Action

Alzheimer's disease, the most common form of dementia, currently has no cure. There are only temporary treatments that reduce symptoms and the progression of the disease. Alzheimer's disease is characterized by the prevalence of plaques of aggregated amyloid β peptide. Yet, recent treatments to prevent plaque formation have done little to relieve disease symptoms. While there have been numerous molecular simulation studies on the mechanisms of amyloid β aggregation, the signaling role has been less studied. Over 38,000 simulated structures, generated from Replica Exchange Molecular Dynamics (REMD) simulations, exploring different conformations of the Aβ42 mutants and wild-type peptides were used to study the relationship between amyloid β torsion angles and disease measures. Six unique methods characterized the data set and pinpointed residues that were associated in aggregation and others associated with signaling. Machine learning techniques were applied to characterize the data and classify how much they influenced the predicted variant of Alzheimer's Disease. Data mining software, such as Orange3, R or Python, provided the ability to use these techniques, to generate lists, and to rank the data. The test and score module coupled with the confusion matrix module analyzed data with calculations of specificity and sensitivity. These methods allowed for developing results in each method of potentially important residues. This research utilizes an algorithm that evaluates all the methods to generate the ten residues that are the most important in characterizing aggregation. These conclusions were developed by analyzing the results and evaluating them based on frequency and rank. Since drugs target specific residues, this research has the potential to guide new drugs to reduce aggregation of amyloid β.

1. Molecular Simulations

Replica Exchange Molecular Dynamics (REMD) simulations were used to explore different conformations of the Aβ42 mutants and wild-type. Because Aβ is intrinsically disordered, the dynamics could not be quantified using global structural changes as with CaM. Instead, the phi-psi angles of the peptide backbone were used as the features to characterize variant-specific dynamics. For each system, N=10 replicas were employed, distributed exponentially from 270 to 600 K. The temperature closest to body temperature, 295 K, was used to assess conformational states of Aβ variants. The wide temperature range was used to enhance conformational sampling. It is difficult to ascertain the connection between free energy states and temperatures because Aβ is a natively disordered peptide sampling manifold of free energy basins. In addition, classic pairwise non-polarizable force fields do not capture well the temperature dependence of conformational ensembles. Therefore, the REMD temperature range serves mainly to generate diverse conformations. Three separate REMD simulations were carried out for each variant and wild-type proteins. Variants were modified using the VMD mutation tool, through deletion and insertion of variant residues into the wild-type Aβ42.

Two preliminary simulations for wild-type and variants were initiated with the PDB structure 1IYT for wild-type Aβ42 and carried out before REMD simulation launches, one to assess the folded state and one the unfolded. To this end, wild-type or variant structures were simulated for more than one nanosecond at 300 K or 600 K. The end PDB structures in the preliminary simulations were used for REMD simulations. Aβ NPT preliminary simulations in explicit solvent had the initial dimensions of the unit cell of 94 Å×93 Å×97 Å and utilized the time step of 2 fs for 20 ns trajectories which was enough time for the calculated RMSD from the initial wild-type structure to stabilize. Minimal changes were observed to the distribution of the ensemble of structures over subintervals at the end of the simulation. Non-bonded interactions were computed using a smooth switching function applied between 10 and 12 Å. Particle-mesh Ewald summation with the grid size of 1 Å was used for electrostatic interactions. Langevin dynamics with the damping coefficient of 10 ps-1 were employed for temperature control, whereas the pressure control was applied by using the Langevin piston method and coupled unit cell dimensions. The production NVT REMD simulations sampling Aβ peptides used the time step of 1 fs. Non-bonded interactions were smoothly switched off in the interval from 7 to 8 Å. Although the cut-offs were utilized for the switching function, which are different from the CHARMM default values, they were applied uniformly for all Aβ simulations including the WT and mutants. Consequently, it is believed that any resulting differences are likely to cancel out when the conformational ensembles of the WT and mutants are compared. The temperature was controlled using Langevin dynamics with the damping coefficient of 10 ps. Three separate REMD simulations were performed for each variant and wild-type Aβ using NAMD default parameter ratios.

2. Data Mining Approaches

The phi and psi angles obtained from the REMD simulations were analyzed using software such as the Orange3 Data Mining Software. The “Rank” modules used to analyze the angles based on their information gain. This is the expected amount of information from the angles. This module works by scoring the variables based on their correlation with variables. The “Test and Score” module analyzes the angles with their ability to account for the data under the ROC curve. For this, 66 percent of the data is the training data leaving 33 percent for the testing. This module required a learning signal. This research used a random forest with 5 trees for this analysis. Finally, the “Confusion Matrix” was used to determine accuracy scores. This allows the research to test the conclusions generates to ensure that the conclusions are accurate.

As used herein, the term “information gain” means the amount of information gained about a random variable or signal from observing another random variable, and thus, can be considered the measure of how much information a feature provides about a class.

3. Training Data Sets

The data set was generated from the REMD molecular simulations as described hereinabove. This data provides over 38,000 simulations of the torsion angles of amyloid β and the mutation and disease they correspond to. The data set can be characterized by either mutation or disease. Both choices have their own benefits.

Benefits of Ranking by Disease: There are only 3 variables that the data needs to be characterized by. Furthermore, it is the final phenotypic expression of the data. This means that it is observable and can be used by the data to make observations and check for errors.

Benefits of Ranking by Mutation: This is a lot more specific as the data now can target many more variables that can be used to determine important residues. Furthermore, it is much easier to determine mutations that aggregate faster meaning the model can account for increased or decreased aggregation mutations to check important residues.

To accurately train the data, the research made each angle a “feature” that can be used to develop the model. Depending on what the data was being trained to test, the mutations and disease were either a “target” or “meta” for this analysis. This ensures that the data is purely characterized by the angles and won't be characterized by the mutations or disease.

4. Results

A series of studies to analyze the contributions of different amino acid residues to measures of functional changes and disease for the different variants. These were done using several machine learning methods to find the phi and psi angles that correlated with the different measures. These measures included amyloid β aggregation, prediction of pathology, drugs targeting amyloid β, and Average Age of Onset. The results are shown with a graph that has rank on the x-axis and residue targeted on the y-axis. This allows for the visual understanding of the general location of the residues. Finally, a confusion matrix was generated to test for sensitivity and accuracy. This ensures that the angles can characterize the disease and are thus important.

5. Prediction Amyloid β Aggregation

The first set of data mining analyses ranked the phi and psi angles of the ensemble of structures based on their ability to characterize measures of amyloid β aggregation in the different variants. The variants studied experimentally by Hatami et al. were separated into two classes, variants that displayed increased aggregation (E22G, L34V, D7N, E22K, A2V, and H6R) and variants that displayed decreased aggregation. The three variants that aggregated the most (E22G, L34V, and D7N) were used for the rankings. The “rank” learning module was used for developing a ranking of phi and psi angles involved in aggregation.

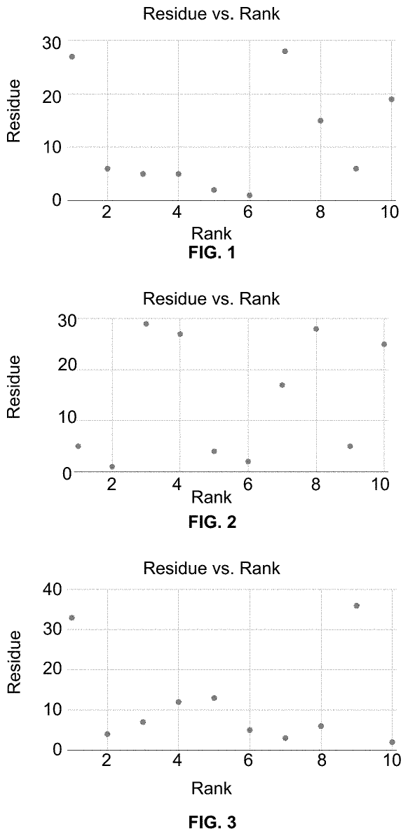

First, the ranking of angles in the variants that cause increased aggregation was compiled. These rankings are illustrated in FIG. 1 as a plot with the rank and residue as the axis. These show how the 5 of the residues in the top 10 were part of the signaling domain. List 1 has the ranking of the angles with their information generated.

Similarly, Yang et al., observed that variants E22G, D23N, and E22Q aggregated faster than variants E22K, A21G, and WT. Both papers are similar in that they both find mutations that aggregate faster, but the mutations they study differ. The research from Yang et al evaluated the mutations that aggregated much faster than the ones that did not. From this paper, it was determined that similar to the last method, this method only ranks the mutations for both increased and decreased aggregation. This research also uses the data from the molecular simulations coupled with the “rank” module.

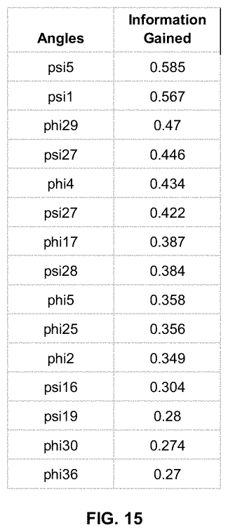

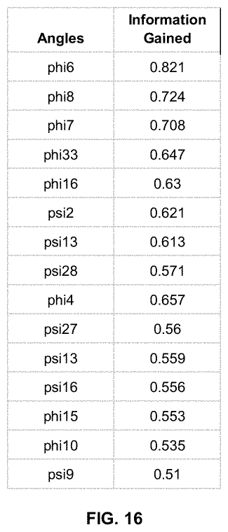

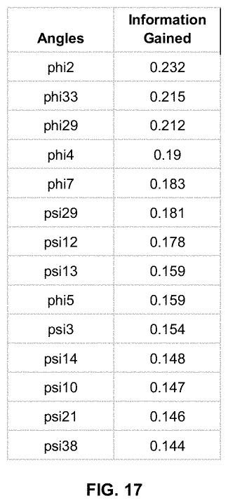

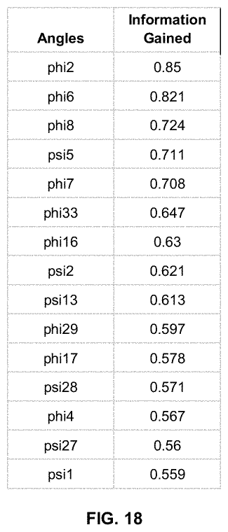

FIG. 2 illustrates the results of a plot of residue versus rank of angles in variants that cause an increased aggregation using a computer-implemented method in accordance with one or more embodiments set forth and described herein. Again, many of the angles ranking high were part of the signaling domain. Based on this method, 4 of the top 10 ranking angles were in the signaling domain. FIG. 15 shows the ranking of the angles along with the information gained from each angle.

6. Prediction of Pathology

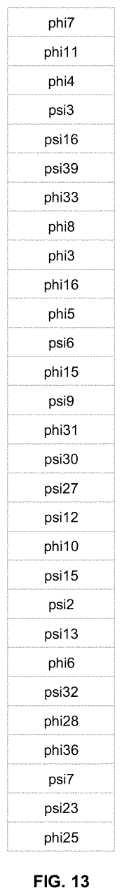

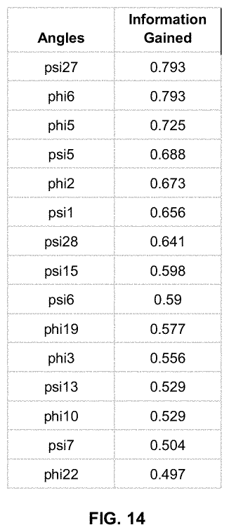

To determine significant residues associated with pathology, an alternative analysis generates a classification tree using the test and score module. The classification tree is a machine learning algorithm that works by dividing the data into subsets. It then predicts the results of the angles in figuring out the target variable with a percent score. The molecular simulation data used was the same data used above. The angles that could separate the WT from the non-WT points by >0 percent were compiled to form a FIG. 13 of angles that are able to predict variants of Alzheimer's (FAD, Familial Alzheimer's Disease and CAA, Cerebral Amyloid Angiopathy) from wild type. These are only the angles that can separate WT from other variants, not FAD from CAA too. These angles indicate a structural change in the amyloid β protein which might result in some functional change that is associated with disease.

FIG. 13 was then used to rank the entire data set by mutation and disease. This is to determine the most useful angles out of FIG. 13. By determining the most useful angles of FIG. 13, the research shows which angles of FIG. 13 are the best at characterizing disease and mutation. FIG. 3 is the result of the ranking of the entire data set by variant of disease based on the angles that could separate disease first. These are the angles that were best able to characterize the data as FAD, CAA, or WT. For the ranking of disease, the ten angles that ranked the best were angles psi27, phi6, phi5, psi5, phi2, psi1, psi28, psi15, psi6, phi19, etc. Again, FIG. 15 has the angles and their information gained generated from this method.

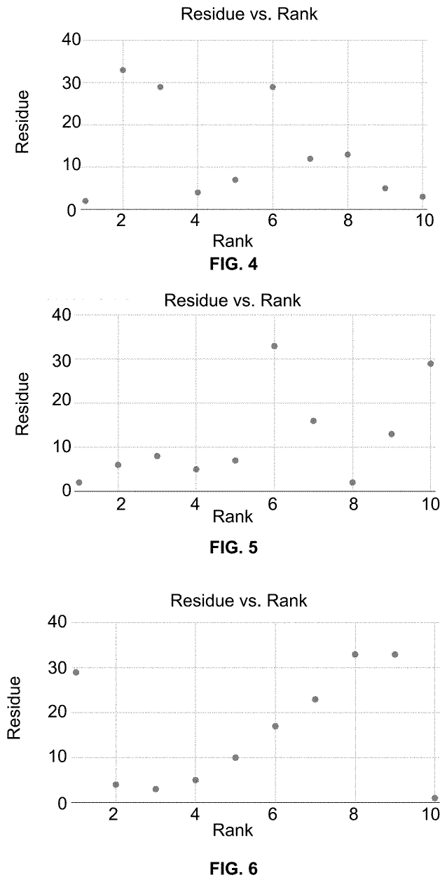

One more method was also applied for this research to predict amyloid β aggregation. This set of data mining analyses ranked the ensemble of phi and psi angles based on their ability to characterize the entire data set based on mutation and disease. These angles must be important because they are able to classify the entire set of angles best, meaning that there is a chance that they can be used to determine the most useful angles at predicting aggregation. This research characterizes both mutations and diseases. This is to ensure that the angles that are the most useful in predicting the important residues are considered. FIG. 4 is the result of the ranking of the entire data set by variant of disease. These are the angles that were best able to characterize the data as FAD, CAA, or WT. FIG. 4 has 5 angles that are ranked as part of the top 10 in the signaling domain. These angles include phi2, phi4, phi7, phi5, and psi3. List 4 has the ranking of the angles and information gained for this method. FIG. 5 is the result of the ranking of the entire data set by variant of mutation. These are the angles that were best able to characterize the data as the specific mutation that causes the disease. This ranking result shows 6 angles that were part of the signaling domain that ranked extremely well. These angles in the signaling domain are phi2, psi2, phi6, phi8, psi5, and phi7. List 5 has the angles that rank the entire data set by mutation and the information gained.

7. Average Age of Onset

The final method looks at the average age of onset for the mutations of amyloid β mutations. With this, there are some mutations that have a significantly faster average age of onset compared to others. This may be related to the aggregation of Alzheimer's disease as mutations with a lower age aggregate faster.

Using this, the mutations that have an average age of onset of less than 60 were analyzed and the 84 angles were ranked based on their ability to influence these mutations. This is important as these angles might be important in the aggregation of amyloid β. These angles were then analyzed to determine the important residues in characterizing mutations with a large average age of onset.

This research tests to see the angles that are significant in characterizing mutations with an average age of onset that are less than 60 years old. The mutations and their average age of onset was found through the paper McCoy et al. This method applied the “rank” module again to test the angles.

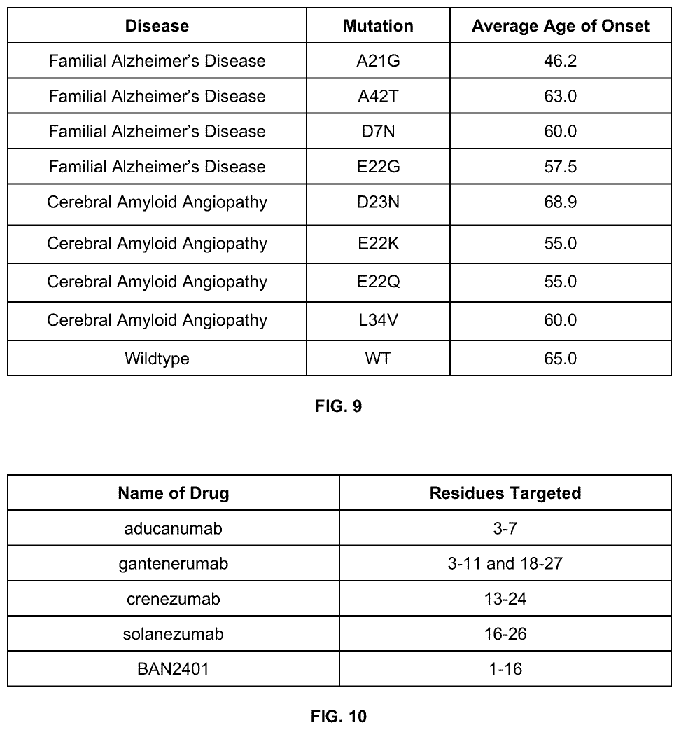

FIG. 9 illustrates the average age of onset along with the mutation. In this, one can see four of the mutations causing FAD and four of them causing CAA. Four of the mutations have an average age of less than 60 years old.

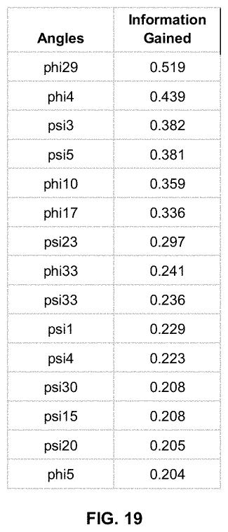

The results are shown in FIG. 6. These are the angles that are best able to characterize the mutations with an average age of onset less than 60 years old. There are four angles that are ranked in the top 10 that are also part of the signaling domain. These angles are phi4, psi3, psi5, and psi1. FIG. 19 has the information gained and the rank of angles for this process.

8. Importance of Location

Evaluating the changes in the phi/psi angles is important in understanding locations where functional change may occur. The use of machine learning to find these functional loci presents a novel approach to identify important sites with applications such as the identification to which effective drugs can be targeted. This research evaluates the phi/psi angles that are best correlated with aggregation as well as those correlated with the signaling domain.

Different loci of the amyloid β structure have different functional implications. Regions of amyloid β such as the hairpin may have importance in aggregation. Our analysis suggests that residues 16-21 and 29-35 are other possible loci where interactions between amyloid β might occur during oligmoerization. Furthermore, other regions such N terminus (residues 1-15) which are thought to interact with small, charged molecules have other functional implications. This is important to analyze to figure out where all the main interactions are and what they can lead to. NMR simulations also show that the changes in the C-terminus increases the chances of aggregation.

Another thing that could be analyzed is the importance of the signaling domain on the aggregation of amyloid β. Having conducted preliminary research, Applicant discovered that there is not a large change in the accuracy when analyzing just the signaling domain compared to just the oligomerization domain despite only testing residues 2-8 where the oligomerization domain tests many more. Using a random forest with 10 trees and without splitting subsets smaller than 5, the test and score modules along with the confusion matrix showed an accuracy of 99.559 percent for the signaling domain and 99.898 percent for the oligomerization domain. This could be a place where additional research is conducted to determine why the accuracy of the signaling domain is so large in comparison to the oligomerization domain.

9. Drugs Targeting Amyloid β

The phi and psi angles involved in drug action that aim to reduce the aggregation of amyloid β were analyzed. These drugs are all currently in clinical trials to test for efficacy. This research only looked at the residues for drugs that were in clinical trials since at least 2018. This is to ensure that the research only looks at recent drugs. This is important because this will result in the highest level of accuracy by virtue of the fact of using the most current research. Furthermore, they have not been proven to fail, yet as the other drugs have.

The drugs target specific residues on amyloid β. At these residues, the drugs do something to stop the aggregation of amyloid β. This must mean that previous research found that these residues were significant. This is because the drugs would not target these locations unless they felt that changing something at these residues would decrease the aggregation. This also means that these residues may provide some functional change at the locations. This is important to ensure that the residues are important in figuring out the significant locations.

These are the drugs that aimed at reducing the aggregation of amyloid β that made it to clinical trials. For the purposes of this disclosure, observations, conclusions, and/or simulations were made based on the drugs that are still in clinical trials. This refers to aducanumab, gantenerumab, crenezumab, solanezumab, and BAN2401. These drugs will have their residues analyzed for these models. The residues targeted were found from past literature available for amyloid β.

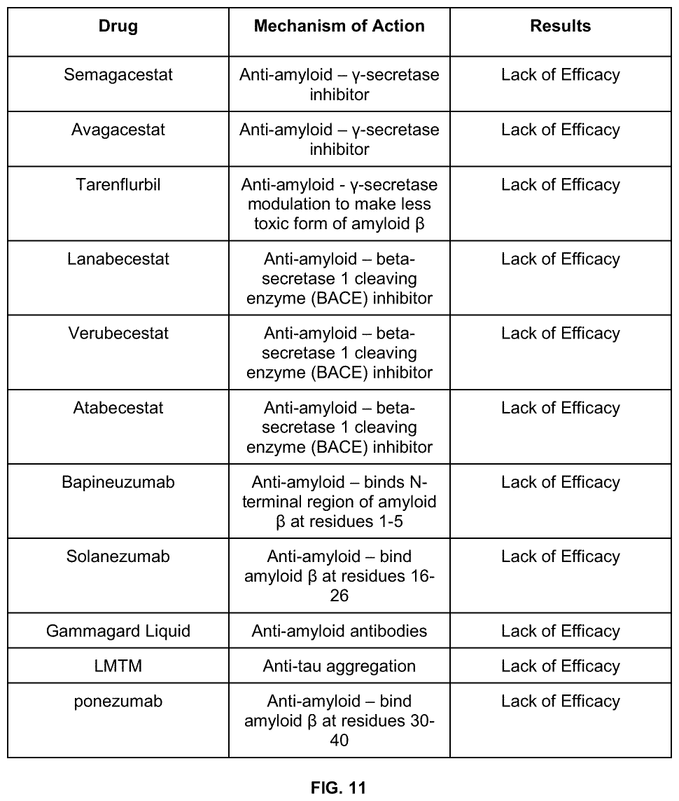

The computer-implemented method in accordance with one or more embodiments essentially analyzes all the residues that are targeted by drugs in clinical trials. This was developed by analyzing the residues targeted by each drug. These residues were then scored based on frequency. FIG. 11 is a table containing the drugs in clinical trials and the residues they target. This was used to identify important locations and domains. As only the drugs in clinical trials were analyzed (aducanumab, gantenerumab, crenezumab, and solanezumab), their residues were also the only parts analyzed. This means that the potentially important residues are in regions: 3-11 and 13-27. All the residues that were targeted by the drugs are in one of these two regions. The other drugs in clinical trials were also analyzed.

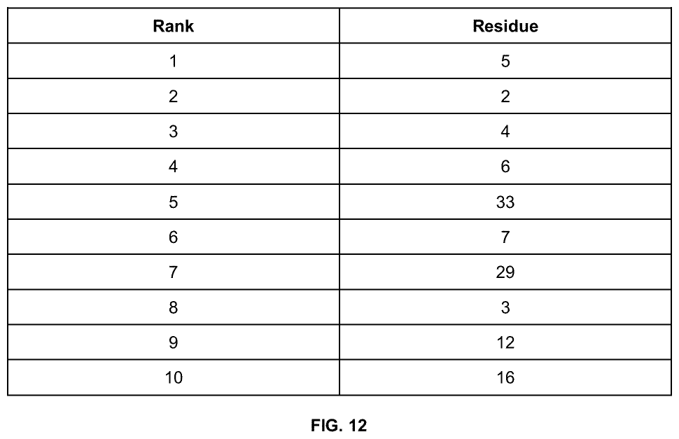

Drugs that failed clinical trials were also analyzed based on their function and their result. The exact residues of these drugs are not publicly available, so no method is able to track the important locations. The drugs that failed, mechanism class, and results have been compiled into FIG. 12. This table can be used to further evaluate the residues if the knowledge becomes public.

10. Bias Evaluation

There is not much bias in this research. This can be attributed primarily to the fact the research is straightforward computational modelling, and thus, doesn't require much room for error. Furthermore, the modeling evaluated the data for not just disease torsion angles, but also the torsion angles for wildtype. This is significant because it reduces the amount of bias in the data generation process. Furthermore, during the process of analyzing the methods, the results were repeatable meaning that there was no significant error involved in the process. The use of six unique methods also allows for any error to balance out. This is because any potential error would be minimized through the weighting process of the different methods. This is another way in which bias is reduced through the process.

These results seem to align with the residues targeted by the drugs that are already in clinical trials. This is important because the researchers who developed the drugs to combat amyloid β aggregation must have looked at research about the residues and locations that are important. As this is the first public research available and this backs up what other researchers have found, it seems to be accurate. Furthermore, all the methods generate similar results despite being unique methods. In fact, each method separately generated at least four of the angles that were determined in the results. This must mean that there is something significant in certain locations that all the methods pick up on.

11. Summary: Data Mining of Molecular Simulations

Based on the results generated hereinabove, for each ranking, the fifteen angles that have the highest rank were given a point value equivalent to their fifteen minus their rank (fifteen minus ranking position). For methods where both increased aggregation and decreased aggregation were calculated, only increased aggregation was used for the purposes of this research. This generates seven unique lists with point values.

For method 5, since residues were not ranked, a new part of the algorithm was utilized. This created a system where every time a residue was included in a clinical trial, it would gain five points. If a residue was repeated, it would gain five times the number of times it was repeated.

These two methods of the algorithm produce scores for residues that were important for each method. From this, the scores are all added up for the final system where it can be determined the most important of the residues. These scores of the residue were then plotted against the residue as a visual showing the residues scores compared to others. The rank of the residue was also plotted against the residue to show how the residues ranked in comparison to each other.

Through this method, the ten residues that ranked best are: 2, 3, 4, 5, 6, 7, 12, 16, 29, and 33. FIG. 12 illustrates the ranking of the residues. The list shows that the residues in the range 2-7, 12, 16, 29, and 33 were the most important from this research.

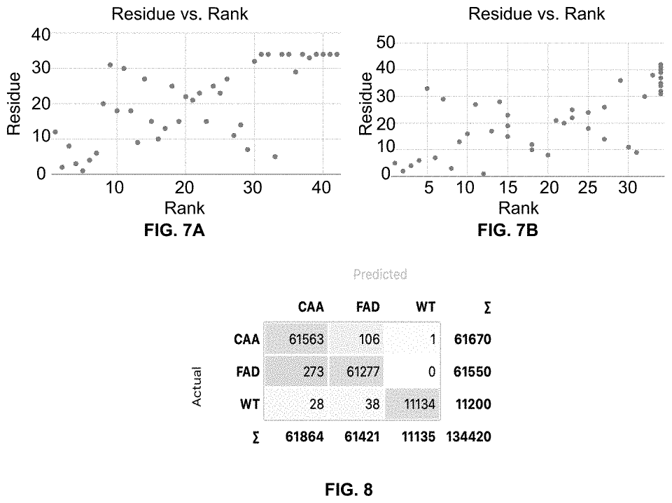

FIG. 7 is the graph of rank vs residues. In this, one can clearly see how in the signaling domain, many of the angles ranked well. One can also see a few more residues ranking well in other key locations of the protein.

FIG. 8 is the significant residues determined by the disclosure on amyloid β. The light green represents the important residues while the dark green represents everything else. This is important in representing the locations that are important. This will allow for the visual understanding of the research conclusions. These residues were then tested with the test and score mechanism, in this example from Orange3 to check for the accuracy these residues have in predicting variants and mutations. From these models, a confusion matrix was developed to check the accuracy of the model.

FIG. 9 is the confusion matrix generated from the ten residues. The total number of correct predictions is 134,064. The total number of incorrect predictions is 446. This results in an accuracy calculation of 99.67%. The sensitivity calculation for FAD is 0.9983. The sensitivity calculation for CAA is 0.9966. The sensitivity calculation for WT is 0.9941. The specificity calculation for CAA is 0.9958. The specificity calculation for FAD is 0.9980. The specificity calculation for WT is 1.0000.

Machine Learning-Based Prediction of Drug and Ligand Binding in BCL-2 Variants Through Molecular Dynamics

The hypothesis for Venetoclax resistance disclosed herein is that changes to the binding pocket can lead to structural restrictions that not only prevent the small molecule binding appropriately, but also allow BCL-2 to retain most of its BH3-binding ability, thereby allowing the maintenance of anti-apoptotic properties. These changes can both be seen qualitatively and measured through structural changes in specific residues. Most importantly, drug resistance can be predicted through molecular dynamics and machine learning.

The structural changes caused by genetic mutations can be identified through simulations of variant protein structures. Analysis of crystal structures of Venetoclax resistant BCL-2 protein variants has shown physical changes within the BH3 binding pocket, and therefore, affect the affinity of drug binding (PDB structures: 600K, 600M, 600L, and 600P). To test the structural changes in other variants, a computational biological approach was taken to facilitate insights into the complicated assemblages of each variant in aqueous solution. Recent advances in molecular computational algorithms, along with the formidable computing capabilities of graphics processing units (CPUs), allow for many stochastic calculations to be performed concurrently over prolonged periods of time. Molecular dynamics simulations generate numerous protein conformations to ascertain the distribution of protein structures at equilibrium. This approach is useful for quantifying small changes within the protein, apart from the elaborate molecular environment. An important challenge in biology is the accurate prediction of the connection and causation between the genetic makeup of an organism (genome) and the totality of all phenotypes or observable physiological traits and characteristics, also known as Genome to Phenome. Genomic variations frequently alter wild-type physiological traits, and for the purposes of the following research these alterations are measured as changes of the phi and psi angles within the protein backbone, changes in the average structure as measured through the molecular dynamics' trajectory, as well as changes in the residue side chains that may affect ligand-binding properties.

Furthermore, structural changes assimilated through simulation trajectories are analyzed and correlated with binding inhibition of Venetoclax to BCL-2. These analyses show that binding inhibition can be predicted through the examination of structural differences using a previously developed computational method, the molecular dynamic phenotype predictive model (MDPPM), which is able to not only predict whether a variant is deleterious but is also able to predict the severity of a disease based on the changes of the dihedral angles, compared to the wild-type. The simulations allow one to analyze the specific changes in the geometry of residues. In the case of BCL-2 changes in the geometry of the binding pocket that may interfere with Venetoclax were examined.

This disclosure shows that alterations in structural features between variants can be used to obtain suitable predictions of phenotypic or pathogenic changes caused by specific variants. In many cases, the information about structure and function is limited. In these circumstances, the MDPPM model can predict phenotypic changes by applying machine learning to the set of phi and psi angles of the ensemble of protein structures for all genetic variants by using molecular dynamics simulations. The computer-implemented method in accordance with one or more embodiments can be used as a broad method for myriad proteins, and in the case of BCL-2 this method was used to predict drug resistance and the effectiveness of the inhibition caused by each variant.

1. Reference PDB Structure and Structural Variant Selection

The original PDB file used for all wild-type simulations, as well as the basis for the variants, was 1G5M. Initially, several variants were used that have been shown to inhibit Venetoclax binding, G101V, G101A, F104C, a “rescue” double variant that restores the binding of Venetoclax, G101V/E152A, and a control for the rescue, E152A. These variants have known Ki and Kd values and already had structural analyses performed. These were used for a comparative structural analysis and for binding groove occlusion measurements. Several other variants where examined that were found in the literature to be Venetoclax resistant. These variants were all used in the MDPPM model for clear-cut predictions of Venetoclax “resistant” or “sensitive” outputs. These included, V92L, D103E, D103Y, D103V, F1041, F104S, F104L, A113G, L119V, R129L, A131V, V156D, T1871, and two negative controls D10E, E179D. Generally, it is thought that E to D or D to E variants in non-conserved regions will cause innocuous changes since both carry negative charge and close in size. Variant structures were acquired by using the VMD mutation tool on the 1G5M structure.

2. Molecular Dynamics Simulation Methods

Nanoscale Molecular Dynamics (NAMD) software was used to generate simulations of wild-type and variant structures of BCL-2. Replica exchange molecular dynamics (REMD) simulations were employed to model the contribution of different conformations of the BCL-2 variants and wild-type proteins to the drug resistant phenotype. For each variant, R=4 replicas were exponentially distributed within a predetermined temperature range of between 310-340 K. Each replica was simulated for 15 ns. Because the upper temperature of 340 K approximately corresponds to the melting point of BCL-2 (˜338 K), the proteins were not expected to unfold within relatively short REMD simulation times. The selected REMD temperature range, however, can facilitate the crossing of energy barriers associated with side chain rotations and minor changes in backbone conformations, thereby achieving equilibrium of the folded state. To verify that replicas perform random walk across temperatures, the average fraction of successful replica swaps was calculated, and found it to be about 0.2. Therefore, the REMD simulations generated an assortment of conformations of the WT protein and its variants.

To perform REMD simulations of BCL-2 and variants, a General Born Implicit Solvent (GBIS) model was used with the time step of 2 fs. All covalent bonds were kept rigid utilizing ShakeH algorithm. The salt concentration was set to 0.3 M, an alpha cutoff was 14 Å, whereas the solvent dielectric constant was equal to 80. The surface tension was selected to be 0.006 kcal/mol/Å². Long-range van-der-Waals and electrostatic interactions were computed every 2 and 4 timesteps, respectively. Non-bonded interactions were smoothly switched off from 15 to 16 Å. Temperature was controlled using Langevin thermostat with a damping coefficient of 5 ps⁻¹. Simulations of BCL-2 utilized the CHARMM36m protein force field.

3. Feature Extraction and Molecular Measurements

The feature sets collected from simulations were investigated using machine learning methods as defined by the MDPPM. Machine learning is practical for molecular dynamics because of the sheer amount of data generated throughout each simulation, and it enables robust algorithms to inspect more aspects of the data by quickly finding key constituents that separate each variable. Using VMD, the dihedral angles of the protein backbone were extracted for the conformations sampled along the trajectories and were used to describe variant dynamics in the prediction models. The raw dihedral angle values of the trajectories were used for all MDPPM calculations and the overall average structure findings. For each of the four replicas in the REMD simulation, of each variant, at least the last 500 frames were used for the dihedral angle extraction and calculations. This assured that the RMSD of the simulation leveled off and that the number of frames met or exceeded the needs of robust statistical analyses. To calculate the average structure throughout all four replicas, each of the last set of PDB files was sequentially loaded into VMD and aligned by the RMSD. Next, the final structure throughout the trajectory was computed via a basic TCL code that computes the phi and psi angles of each file in the new DCD trajectory and averages them as it traverses the trajectory.

Molecular measurements were conducted by determining the distance between R146, which is in a highly conserved region known as the NWGR motif, and the Y108 residue, which is in the conserved BH3 region and located directly across the binding groove from R146. The NWGR motif is part of the BH1 domain in the BCl-2 family and is important for the suppression of apoptosis. This sequence produces an N-terminal helix-capping motif at the initiation site of helix α5 and is highly conserved in the cl-2 family. For each average structure, the distances between Y108/OH and R146/CA, 108/OH and 151/C, 110/CA and 143/CA, 109/NH1, and 147/C were used for the α2 to α5 measurements across the binding groove and calculated using PyMol on the subsequent average trajectory structures for each variant.

In-silico empirical data was correlated and compared to X-ray crystallography for the structural analyses and surface plasmon resonance (SPR) for kinetic measurements of the experimentally determined Kᵢ and K_{d}. It is unclear precisely how SPR measurements relate to the clinical concentrations of Venetoclax in cells given the complexities of pharmacokinetics. The inhibition ratios between each variant compared to the wild-type can be evaluated, however, and the SPR assay is highly sensitive. The evaluated resistance level ratio comparisons can likely be used to assess and predict clinical resistance.

Computations for Euclidean distances were attained through calculations of the dihedral angles throughout the trajectory using a principal component analysis. Euclidean distances were established by calculating the distance between the centroids of each principal component as compared to the wild-type. The distance for BIM and BAX was calculated from a Principal Component Analysis (PCA) on all phi and psi angles whereas the Venetoclax calculations only utilized psi94 to phi114, based off the observation that these key residues shifted most between the average variant structures.

4. Prediction Testing

Predictions for Venetoclax susceptibility utilized the random forest algorithm to predict a “resistant” versus “sensitive” classification of variants. The exact code and libraries necessary to execute the MDPP were based on those from github.com/MDPPM/initialCode. Briefly, to do this, RStudio was used to execute the principal component analysis, then a subsequent random forest algorithm on the principal components to evaluate which are the most accurate for classification. Subsequently, a random forest predictive model, K-nearest neighbours, or other machine learning module can then be used on the most accurate principal components (PC's) to classify on a “sensitive/resistant” basis. Here, random forest was used in both cases to locate the accurate PCs for training data and to predict whether a variant was Venetoclax sensitive or resistant. To control for biases in the training data, the testing variant was removed so that there was no sampling or training using the test data, trained the random forest algorithm on the remaining variant or wild-type dihedral angles, then added back in the test variable for classification. Further, an aim was for the end-user to be able to design a model that can train data that skews more towards a false positive or false negative prediction, depending on the desired outcome. This was accomplished as well as demonstrate accuracy and plasticity by utilizing a different set of accurate PCs for the random forest training.

Several tools have been developed to predict if a particular missense mutation will cause a pathogenic change in function. These tools include, but are not limited to, SIFT, Polyphen-1, Polyphen-2, MutationAssessor, FATHMM (Functional Analysis through Hidden Markov Models), PhD-SNP, SNAP, PANTHER, Auto-Mute, PLINK, CC/PBSA, and I-Mutant. Several consensus tools, such as PON-P, Condel, and PredictSNP have been created to combine multiple tools to get a better prediction of phenotype using the default settings. These tools generally classify a variant as non-pathological or pathogenic, with some allowing an intermediate classification of possibly pathogenic. These tools were used to predict varriants of 1G5M as a comparison to the MDPPM predictions. These tools to do not specifically predict drug resistance, however, it has been suggested that they might be appllied for such purposes.

5. Variant Classification

The PC's for the ensemble of phi and psi angles for the variants was also calculated using the Principal Component Analysis workflow example in the Orange3 software version 3.26. The Classification Tree example workflow was used to see which PCs were sufficient to classify the data.

6. Results: BCL-2 Variant Modeling

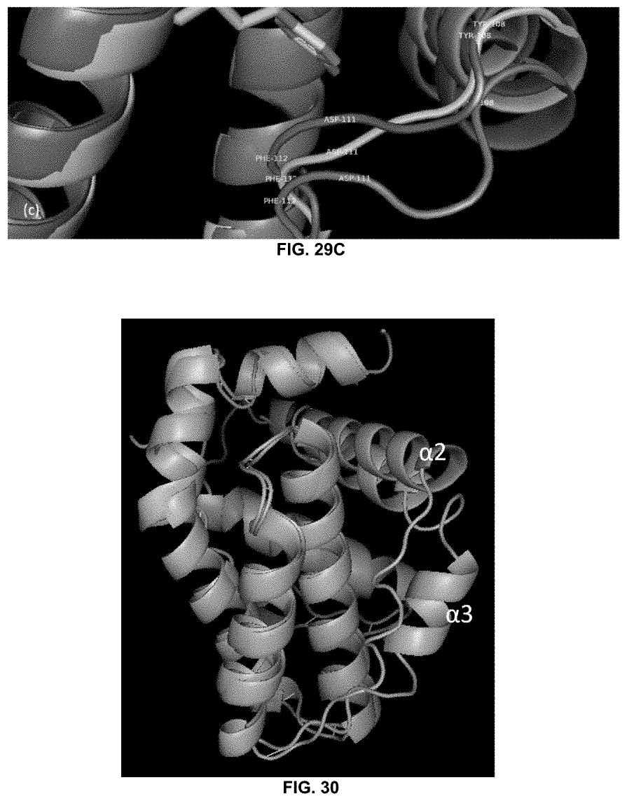

The initial phase of this study was designed to serve as a foundation for accuracy of measurements as well as to validate the remainder of the investigation and subsequent findings. Initially, the goal was to recapitulate the X-ray crystal structure established in previous studies through molecular dynamics, with the exception of using an unoccupied BCL-2 protein, one that was not bound to Venetoclax or any other proteins. The results of the unbound average structures throughout the trajectory for G101V and WT were compared to the previously studied X-ray crystallography assemblies bound to Venetoclax. The previous studies found that the G101V mutation causes a knock-on effect of an adjacent amino acid, E152, which results in a sidechain rotamer of approximately 60 degrees for the residue. It was also documented in this study that the G101V variant bound to Venetoclax causes a large shift of the Y18 side chain. FIGS. 29A through 29C demonstrates that our simulation findings show analogous effects to both the E152 knock-on rotamer in the Venetoclax-bound model, as well as the changes in Y18, but that the E152 rotamer is observed to be approximately 45 degrees pivoted in our model as opposed to the previously observed 60 degrees (FIG. 29B).

In FIG. 29A, space filling model of the R-chain shifts that were also found in X-ray crystal structures of E152 and Y118. The V101 residue adds atomic mass where there was not any before because of the glycine which, in turn, causes the knock-on effect and cause these proteins to move. In FIG. 29B, the angle of rotation in wild-type (green) and G101V of E152 (cyan) that is analogous to X-ray crystallography work. In FIG. 29B, WT (green), G101V (red), and the rescue variant G101VE152A (blue) are illustrated. As detailed, past evidence shows that the G101V variant drug resistance can, in practice, be “rescued” through the synthetic double variant. The data confirms this and displays why this is likely. The D111 and F112 residues inserted into the pocket by G101V are pulled back out in the rescue and allows for the chlorine of Venetoclax to be able to push through. Although the Y108 molecule is rotated, which can also obstruct the binding groove, it is also translocated down and out of the way.

An overall qualitative view of the average, unbound BCL-2 WT structure found in our simulations overlaid with those found in the crystallography study bound to Venetoclax can be seen in FIG. 30. Notably, there are changes in the α2 helix where Venetoclax binds.

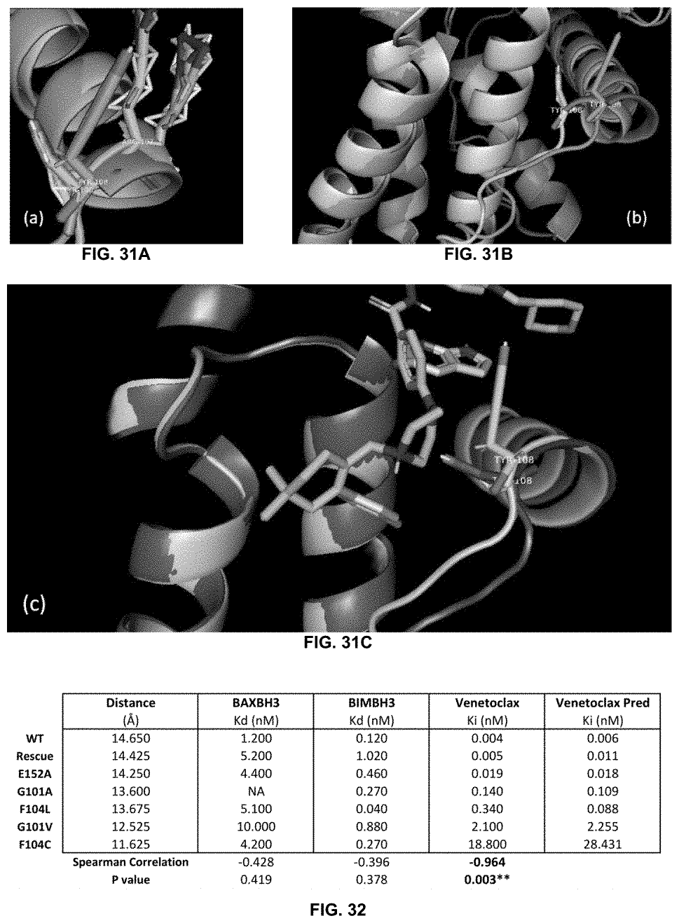

In addition to corroborating previous in-vitro data of the Venetoclax-bound structure, in our unbound structure, it was observed that residues D111 and F112 were shifted into the binding pocket in the G101V variant (FIG. 29C), an observation that was not revealed in a previous study. Further, it was uncovered that the G101V variant had at least two other residues alter location relative to the wild-type, R107 and Y108, both of which drastically shift, and both of which interact directly with the Venetoclax molecule (FIGS. 31A through 31C). In FIG. 31A, WT (green) and G101V (blue) showing Y108 and R107 side chain rotation. FIG. 31B illustrates F104C and the Y108 rotamer, FIG. 31C illustrates rotation of Y108 in G101A with Venetoclax bound from AutoDock. Past evidence also shows that the G101V variant drug resistance can, in practice, be “rescued” through a synthetic double variant G101V/E152A. The data confirmed this through quantitative measurements and establishes why this is likely the case. The double variant causes the D111 and F112 residues that were inserted into the pocket by the G101V variant to be pulled back out, leaving a pocket that is not the same as, but is at least analogous to, the wild-type (FIG. 29C). The change in distance was measured for D111 and F112 between the WT and G101V to be 2.5 Angstroms and 2.9, respectively. The distance between the Rescue and WT was −0.4 and −1.1 Angstroms for D111 and F112, respectively, denoted as negative because they extend in the opposite direction and possibly lead to an increased pocket area.

Various distances were measured from the residues that exist transversely to each other in the binding pocket. When gaged, it was found that these distances correlated to Venetoclax inhibition detected by SPR data. Observations by Applicant revealed that the residues at the end of the α2 helix, 107-109 were located close to the binding groove and were the residues that were most likely to shift between each of the structures. The distances of these residues were measured to the conserved residues on α5 and compared the extent between variants (FIG. 32). These expanses between the α2 and α5 pocket residues were not found to be significantly correlated to BAX or BIM Kd values, however, were correlated to Venetoclax inhibition. The Spearman correlations for BAX and BIM were measured to be −0.43 and −0.40 respectively, with p values greater than 0.35, while Venetoclax was −0.96 with a p value of 0.003 (FIG. 32).

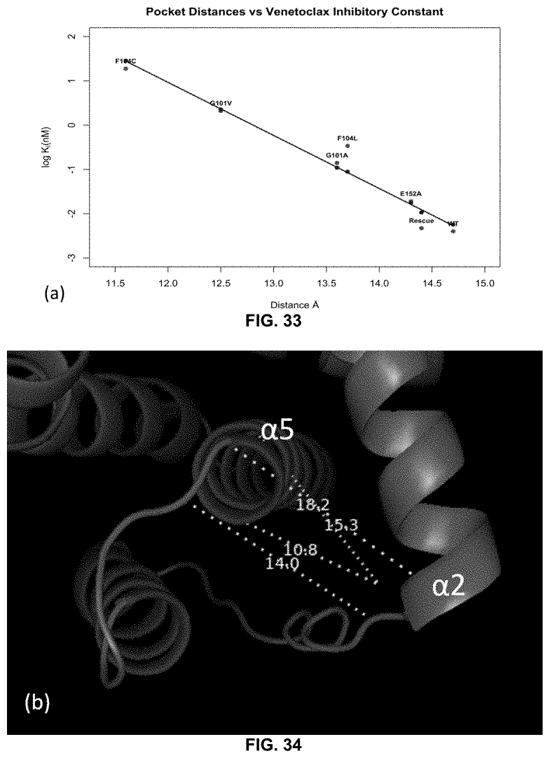

FIG. 33 shows the Venetoclax predicted values of the Ki found by linear regression versus the distances. Venetoclax predicted values versus real values had a Pearson correlation of 0.999 with a P value of 1.283e-08.

FIG. 34 illustrates an example of the distances measured in Angstroms across the binding channel in the wild-type protein. These same distances were measured for all variants and the average of the four was used to indicate differences between variants.

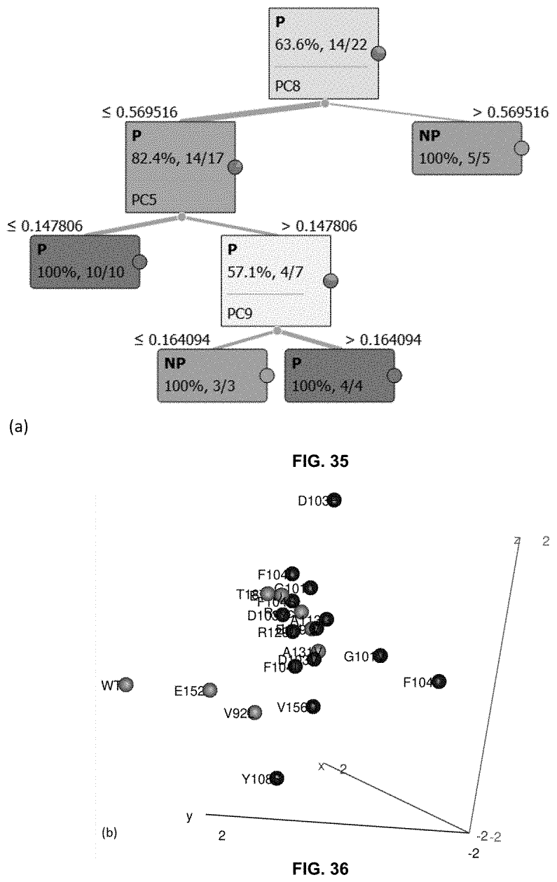

A classification tree can correctly separate 100% of the variants using PC5, PC8 and PC9 (FIGS. 35 and 36). In FIG. 35, the classification tree completely separates the drug resistance from drug sensitive variants using PC5, PC8 and PC9. In FIG. 36, a 3D scatter plot of using these three dimensions shows separation of the variants as drug resistant (blue) and drug sensitive (green). This indicates that there is adequate information in these three dimensions for classification. Using these three PC dimensions, a 3D plot shows the separation of the drug-resistant from the drug sensitive variants (FIGS. 35 and 36).

Universal predictions of each variant were classified using various predictive models. These methods did not classify variants as “Venetoclax resistant” or “Venetoclax sensitive” but rather made the classification of “deleterious”, “unknown”, or “neutral,” respectively, using the MDPPM as well as other current computational tools for comparisons.

The results show that the advantage of the MDPPM model is its utility to be able to evaluate more than one variant at a time and with greater than 90% accuracy using a leave-one-out model for classification. Other models were either not as accurate, unable to be geared towards the desired outcomes of false negatives or positives, or not able to perform on a double variant.

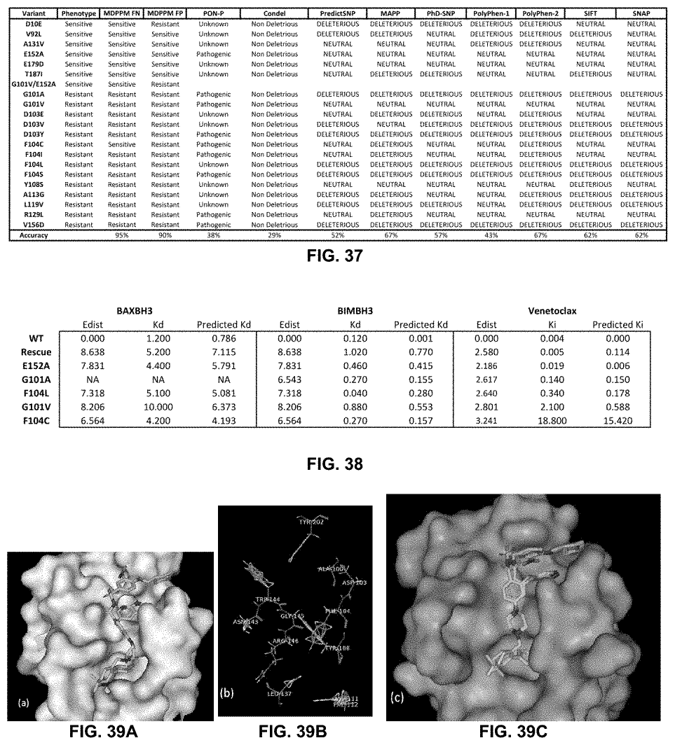

FIG. 37 shows the results of the MDPPM and other predictive software, showing the predictions of each model and comparing the MDPPM model for BCL-2 to other software programs. FP and FN are the modes of the model that were trained for the two best outcomes. One had two false positive outcomes and the other had a false negative outcome. Other software shows varying degrees of accuracy under the assumption that pathogenic is equivalent to a change in function that might suggest drug resistance. The scores in the table were returned by the software and the meaning is indicated by the designation “deleterious” or “non-deleterious” which is also synonymous to “neutral.” The “unknown” classification by PON-P was not classified. The experimental/clinical observation of pathogenicity versus non-pathogenicity in the scientific literature and databases such as uniprot and ClinVar is used for the “ground truth” for classifying variants.

The MDPPM method correctly predicted pathological vs non-pathological with 95% accuracy using a designed false negative method, or, 90% accuracy using a false positive approach, compared to the other methods that contain varying amounts of error. Furthermore, the other methods were not able to differentiate between different disease phenotypes caused by the variants of a particular gene, whereas the MDPPM method does this accurately. Finally, the MDPPM method can predict the severity of the phenotype as shown by the high correlation coefficients comparing the distance from WT to the different measures (FIGS. 32 and 38). In FIG. 38, the distances for Venetoclax were measured using only amino acids found to interact with the molecule. BIM Kd predictions compared to the actual values had a Spearman correlation of 0.77 and a P-value of 0.04. For BAX K_{d}, a Spearman correlation of 0.89 with a P-value of 0.03 and for Venetoclax, a Spearman correlation of 0.96 with a P-value of 0.0014.

To get a better idea of the specific residues that bound to Venetoclax in the model derived in accordance with one or more embodiments, AutoDock Vina was used to visualize docking, or what was presupposed to be a very close initial approach by Venetoclax, to the unbound molecule derived from the simulations. It was found that mostly hydrophobic residues interact with the Venetoclax molecule, and it was discovered to be somewhat analogous to the X-ray crystal structure, PDB ID 6O0K (FIG. 30).

FIG. 39A shows Venetoclax bound to simulation-derived wild-type BCL-2 protein as instituted through AutoDock Vina. FIG. 39B shows the residues are found mainly to interact with Venetoclax in accordance with the computer-implemented method of one or more embodiments. FIG. 39C shows BCL-2 bound to Venetoclax structure of PDB ID 600K. FIGS. 39A through 39C show that Venetoclax is in close proximity to Y108, R146, and G145, and the structure of Venetoclax binding resulting from Autodock on our average model was similar to the one derived from X-ray crystallography with the exception of the α2 helix being lowered in the crystallography structure and the exact positions of the Venetoclax molecule are not in the same conformation in several locations. However, X-ray crystal structure may contain artifacts from the crystal packing and the stochastic nature of AutoDock Vina should be considered.

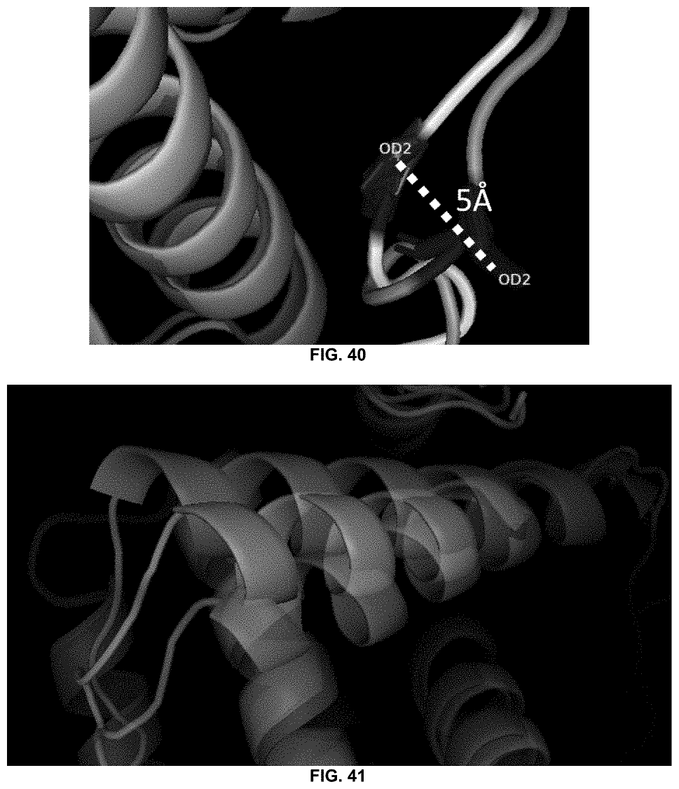

For each variant, there was at least one residue that had either shifted positions or rotated (some not illustrated). In F104L, again it was observed that D111 moved into the pocket, in which FIG. 40 shows an F104L (white) 0D2 atom of residue D111 closer to adjacent α5 helix by 5 Angstroms.

In another example, E152A, the α2 helix was drastically shifted lower in the variant (FIG. 41) as compared to wild-type, and the double variant rescue G101V/E152A also displayed α2 helix lowering, but not as drastic. In FIG. 41, E152A (cyan) exhibits α2 helix downward shift as compared to the wild-type (green). The double variant G101VE152A (purple), the α2 helix is also lowered but not as drastically.

7. Results: Variant Classification

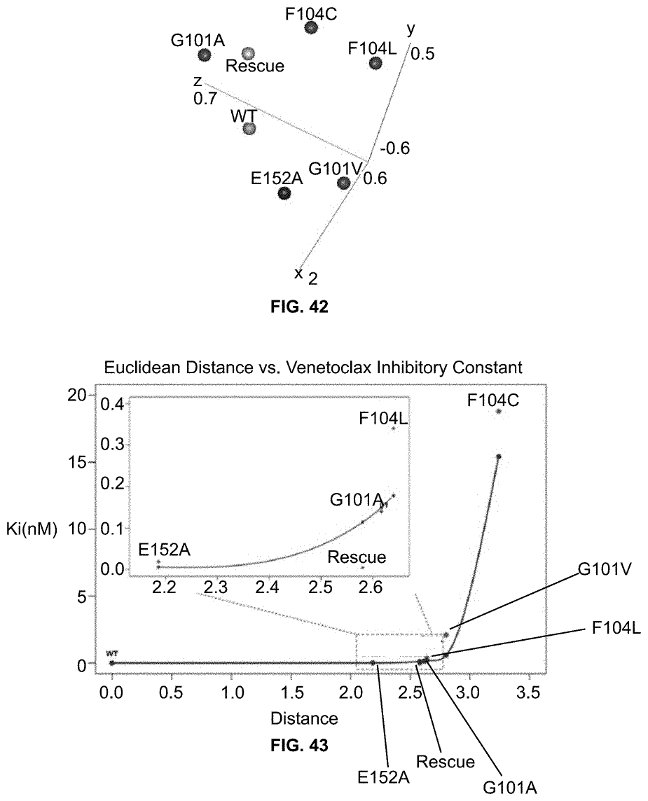

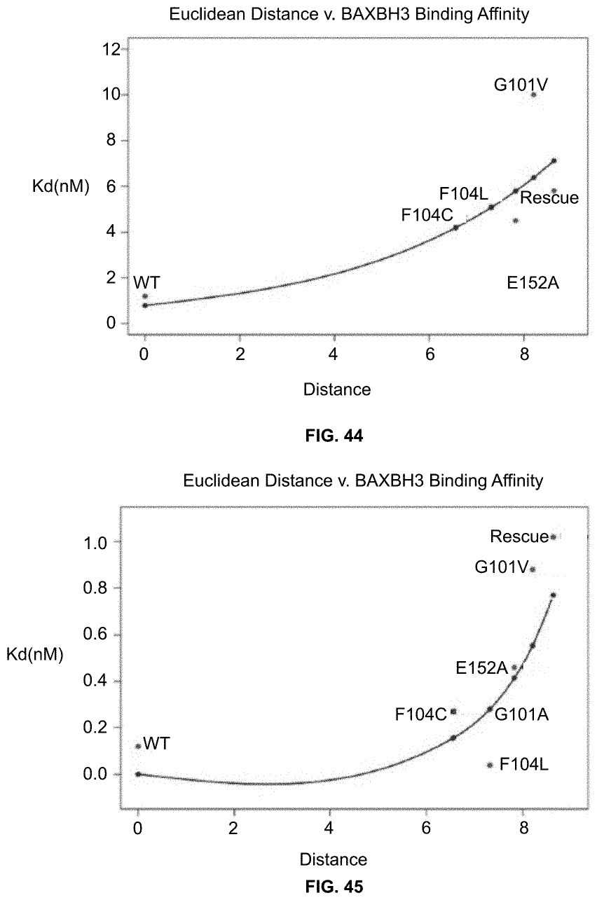

Euclidean distances were compared to the Kd values for BAX and BIM, and the Ki values for Venetoclax (FIGS. 42 through 45). FIG. 42 shows the separation of the variants and their Euclidean distance from wild-type. FIG. 43 shows the MDPPM method used to predict the severity of drug resistance used on the smaller subset of dihedral angles specific for Venetoclax. FIGS. 44 and 45 show BAX and (d) BIM regression predictions, with predicted values shown as blue points, actual predictions shown in red.

Data predictions were made for K_{d} and Kᵢ by utilizing linear regression from the fitted line between the Euclidean distance versus log(K_{d}) or log(Kᵢ) and non-linear regression for the fitted line between Euclidean distance versus K_{d} or Kᵢ. The Kᵢ values of Venetoclax compared to the regression fitted predicted values had a Spearman correlation of 0.96 with a P-value of 0.0014, and for the BIM K_{d} a Spearman correlation was calculated as 0.77 and a P-value of 0.04. For BAX K_{d}, a Spearman correlation of 0.89 with a P-value of 0.03 (FIG. 32).

8. Discussion

The establishment of rapid sequencing technologies has developed the ability to sequence genomes and discover gene variants at an unprecedented rate. A major remaining challenge is the phenotypic or functional classification of these newly discovered variants as well as a lack of positive controls. Currently, many variants are classified as variants of unknown significance (VUS) in the numerous databases. The other software tools used herein to assess whether a variant is deleterious or non-deleterious failed to predict the severity of variants or allow for the classification of multiple variants in the same gene. The MDDPM proved an in-silico method that addresses these issues and can be tailored to each desired scenario.

Overall, in the field of cancer, many variants have been associated with causing the dysfunction underlying the cancer or conferring resistance to cancer therapy drugs or both. One of the major causes of failed cancer drug treatment is drug resistant variants. The American College of Medical Genetics (ACMG) has set the standards for the classification of variants and, currently, in-silico methods are not recommended during classification without other corroborating evidence. This is due in part by an inability of the present-day tools to differentiate between a cancer-causing variant and a drug-resistant variant. Instead, these current tools can only make a prediction about pathogenicity, i.e., the decision is based on whether a variant will change the normal function of the protein, without the specification of the exact alteration.

Secondly, these tools are trained to predict such pathological behavior in a plurality of protein targets. On the other hand, MDPPM is specifically trained, in accordance with one or more embodiments, on the variants for each protein targeted by the cancer drug. The application of the MDPPM method presented is the first step toward an accurate and precise way to make such determinations. A leave-one-out classification model trained on twenty-one variants was used to mimic the real-world situation of finding an unknown variant, and training on all the existing data before classification of the unknown variant. The Clinical Genome Resource (ClinGen) Sequence Variant Interpretation (SVI) Working Group has recommended that there be a minimum of eleven total pathogenic and benign variant controls to reach moderate-level evidence for variant classification. For now, the safe conclusion is that the MDDPM can classify drug resistance or susceptibility to Ventoclax for BCL-2 variants. Further studies may be needed on additional drugs and targets to make broader assertions.

The simulation studies showed that shifts in the conformation of residues that partially obstruct the Venetoclax binding groove contribute to binding inhibition. In the G101V variant, the D111 and D112 residues moved upward into the groove. Applicant has hypothesized that this happens because of the D111 and F112 positioning within the molecule, which is situated in a place that is more malleable than other key residues in the protein due to the glycine at position 110. The positions of D111 and F112 found in accordance with this disclosure were not instituted in the X-ray crystallography data. It is believed, however, that the conformation found in these previous studies do indeed occur in the unbound protein, especially because of the flexibility due to the glycine. But that the probability of D111 and F112 being in this position is orders of magnitude lower than where they are located on average, which is the conformation obtained from the present simulations and the reason that the Ki is so much higher. While there is not a perfect agreement between the simulation and X-ray structures, there are many reasons this could occur such as high sample concentrations and crystal packing vs. the aqueous environment that is mimicked in the simulations. In addition, X-ray crystallography protein structures obtained may not be in a completely “relaxed” state.

In addition, the properties of the variants showed some other thought-provoking properties that allow one to speculate on the specific mechanism of Venetoclax resistance. In E152A, for example, it was found that the α2 helix was drastically shifted lower in the variant as compared to wild-type. This is most likely caused by the diminished R group size of alanine from glutamic acid. The α2 helix rests on, or by some means needs the bulk of the alanine in the wild-type structure, and this acts as a scaffold which is absent in the E152A mutation. This hypothesis is bolstered by the results of the double variant structure. When removing the bulky R group of glutamic acid in G101V to G101V/E152A, this too showed that the α2 helix was lowered, but not as drastically, and conceivably this is because of the added mass of the valine from glycine. It is noted, however, that in both instances where the α2 helix was lowered, the binding affinity of Venetoclax was not inhibited and that the helix is shifted with respect to the crystal structure, so the lowering of the helix is most likely of no consequence. When the bound and unbound protein were overlaid, it was observed that the α2 helix was lowered there as well and may be a conformational change when Venetoclax binds.

Although the α2 helix downswings in E152A and G101V/E152A look dramatic, it was found that what correlated most to Venetoclax inhibition structurally was the distance between the residues that sit across from each other in the binding pocket. It was found that as the end residues of the α2 helix, 108, 109,110, become closer to the conserved residues of the α5 helix Venetoclax inhibition goes up. Given all other changes, it may be that these residues, at least in terms of Venetoclax binding, are the gatekeepers, or the most important residues that need to be kept clear of the binding groove. Evidence of this stems from the dramatic resistance of the F104C residue, that has no other major shifts in structure except for these residues moving so substantially into the binding groove.

Further, it was demonstrated that there is a difference between a general BH3 binding to the groove and Venotclax-specific binding, and it was exhibited how that comes to fruition. That is, the distance between the key α2 residues and the α5 helix was not significantly correlated to BH3 binding but was to Venetoclax. This indicates that Venetoclax inhibition can occur, while at the same time, the affinity for BH3 binding remains the same. And this also suggests that these amino acids are exceptionally important for Venetoclax binding.

9. Summary

Molecular dynamics provide a feature set for the application of machine learning to predict the phenotypic or functional changes of genetic variants of BCL-2. In the case where there is specific knowledge of structural features, for example, the dimensions of the binding pocket, accurate predictions are possible. In other cases where there the protein is less well studied, however, using the phi and psi angles of the amino acid backbone allow for accurate prediction. It was shown in the case of BCL-2, specifically, the precise residues that impinge upon the binding of Venetoclax can be identified/located. From this, one can envision other rescue protein development and testing, perhaps second stage BH3 mimetics research that consider the effects that each of these variants cause to the binding groove. Further, predictions were made about the classification of specific variants. The computer-implemented method in accordance with one or more embodiments was not only able predict that a variant would be Venetoclax-resistant, but also detect how much the variant modulated the Kᵢ of Venetoclax or the K_{d} of BH3 binding.

Peptide Inhibitors of Sars-Cov-2 nsp10/nsp16 Methyltransferase Predicted Through Molecular Simulation and Machine Learning

This study had the goal of finding and proposing new analogous nsp10 peptides that can bind nsp16 with equal to or higher affinity than those naturally occurring. The research demonstrates that in-silico molecular simulations can shed light on peptide structures and predict the potential of new peptides to interrupt methyltransferase activity via the nsp10/nsp16 interface. A new set of peptides was developed by Applicant based on conserved regions of the nsp10 protein throughout the Coronaviridae species and test these to known MTase variant values and present two solid new candidates for potential new testing. It is envisioned that these new leads are the beginning of a reputable foundation of a new computational method that combats coronaviruses and that is beneficial for new peptide drug development.

1. Reference Protein Data Bank (PDB) Structure and Structural Variant Selection

The reference file used to derive peptide sequences was located at the protein databank of The Research Collaboratory for Structural Bioinformatics PDB, PDBID 6ZPE for SARS CoV-2 nsp10. The molecule was removed of all residues excluding F68 to Y96 and used for simulations. Variants were introduced into the new peptide via VMD mutator tool and initial mutations were chosen from the literature where they were shown to cause changes in methyltransferase activity of whole protein interactions of nsp10 and nsp16. These variants were G70A, G94A, G94D, H80A, K93A, K95A, R78A, R78G, S72A, Y96A, Y96F, Y961, Y96V. New variant sequences were selected due to their occurrence in semi-conserved residues at the sites F68 to Y96 in the Coronaviridae subfamily. The foundation of this hypothesis being that the Y96F variant increased methyltransferase activity in the literature and had occurred in other virus sequences in the subfamily. The new peptide variants chosen were C731, C73V, C79A, F68Y, H80R, K93R, L92F, L92Y, Y96C, Y96 W.

2. Molecular Dynamics Simulation Methods

Molecular dynamics simulations were deployed by using Nanoscale Molecular Dynamics (NAMD) using the CHARMM36 forcefield. The phi and psi angles of the simulations were extracted by using VMD followed by a principal component analysis (PCA) using RStudio (RStudio Team, 2021) that was used to find the Euclidean distance measurements on the resulting principal components (PCs). Euclidean distances were calculated by measuring from centroid to centroid of the PCs from the wild-type. The Euclidean distances were correlated to previous methyltransferase data by using a Pearson's statistical test in R. Three simulations were carried out for each peptide at 310K due to the instability of each variant peptide. It was found that at higher temperatures the stabilities varied greatly, and some structures fell apart. All simulations were carried out for more than 20 nanoseconds as the peptide was taken from the larger protein and therefore folding assessments with longer simulation times were not necessary. The General Born Implicit Solvent GBIS model was used for all simulations with a 2 fs timestep. ShakeH algorithm was set to “on,” salt concentrations were set at 0.3M, and the surface tension was set to 0.006 kcal/mol/A, with Langevin dynamics used for temperature control.

3. Feature Extraction

Utilizing VMD, the phi and psi dihedral angles of the protein were extracted for the desired timepoints used in the analytical calculations. A set of raw dihedral angle data for each variant were copied into a matrix and utilized for all MDPPM computations. For at least two of the simulations, greater than 500 of the last PDB file frames were placed into the matrix and used for machine learning or PCA analysis. Using the last 500 frames allows for two things, one, the root mean square deviation smooths out in the trajectory, which indicate that the peptide has stabilized, and two, the number of frames is enough for vigorous statistical analyses. The average peptide structures of the simulation trajectories were found by averaging the frames by using a small TCL code. Average structures were viewed qualitatively for fit and structure changes or atomic distance measurements were viewed or taken via PyMol (Schrodinger, LLC, New York, N.Y.).

To fit the peptide variant data to methyltransferase activity, nsp10/nsp16 interaction and methyltransferase data were collected. The empirical data was taken from Bouvet et al. and is gathered by measuring in-vitro interaction assays, % BRET, which measures energy transfer and is used as a proximity-based assay to monitor protein-protein interactions and conformational rearrangements in live cells, and 2′-O-MTase activity. The Euclidean distances calculated from the simulations were correlated to these data for comparisons to methyltransferase activity and the structure of the peptide. The Euclidean distance measurements was correlated to percent BRET, percent Interaction between nsp10 and nsp16, and percent Activity 2′-O-MTase (referred hereafter also as MTase or methyltransferase).

To predict if a new variant would have the same, or possibly increased, MTase activity, a random forest algorithm was used. Initial tests used only the variants that had in-vivo or in-vitro data to qualify and verify method success, which was a strong correlation to previous data from the PCA analysis. To reduce bias in the training data and to mimic clinical settings for unknown variants, the variant to be tested out was pulled, trained on the remaining variants, and the new variant was added to the already trained data to assess predicted classification. Only variants with data were used for training and subsequently classified as having “low,” “middle,” or “high,” MTase activity based on the MTase data. Any variants with lower than 40% was classified as having “low” activity, 40-80% as “middle,” and >80% as “high.” After obtaining satisfactory results with the prediction model on the variants with data, the newly derived peptide variants were then added in from the natural product research. Only those new variants classified as “high” were categorized as potential new candidates for future research. The model was developed to take a false positive approach, where the variant was deemed more likely to cause a decrease in MTase activity, so that as to error on the side of caution when screening new variants.

4. Results

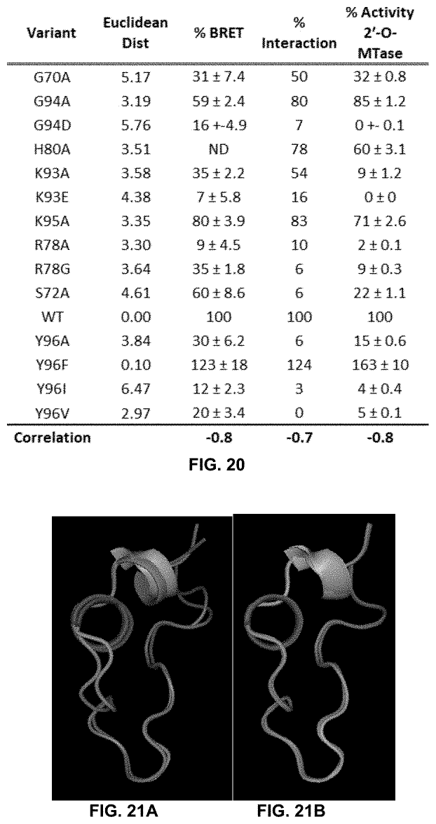

The first step in predictive modeling and assessment using the MDPPM is an all-atom simulation of the protein followed by a PCA analysis on the phi and psi dihedral angles. Euclidean distances are then measured between the variants and the wild type. FIG. 20 illustrates the Euclidean distances calculated from the phi an psi dihedral angles and correlates those to the Bouvet et al (2014) data. These data show that the model results strongly correlate to all three endpoints with statistically significant P-values using Pearson correlations. The results for the nsp10 peptides show a correlation of the Euclidean distances to % BRET, % Interaction, and the % Activity of MTase to be −0.8, −0.7, and −0.8 respectively, as well as P values of 5.5×10⁻⁴, 1.5 10⁻³, and 4.5×10⁻⁴, respectively.

Next, the average structures of each peptide throughout the simulation were examined. When comparing with the current literature, there are conserved regions in the region of nsp10 where the peptides were extracted and designed the experiment to first compare the conserved regions of the wild-type peptide to the variants. Initially, a comparative analysis of the peptides was performed using a general alignment in PyMol. Initially, a qualitative examination of the structural differences of the peptide variants that had the largest and smallest Euclidean distances as compared to the wild-type peptide to gauge structural dissimilarities and similarities, those variants were G94D and Y96F. G94D is one of only two variant peptides that resulted in zero methyltransferase activity and the Y96F variant increased this activity.

FIG. 21A illustrates a wild-type (green) and G94D (magenta) overlay displaying large differences in several positions, particularly, F68, H80, 181, G94D, and Y96. FIG. 21B illustrates a wild-type (green) and Y96F (cyan) overlay exhibiting nearly complete structure homogeneity with a few subtle differences. These figures show that the G94D average structure did not fit well onto the wild-type protein. Most notably, the illustrations reveal misalignments at residues F68, H80, I81, D94, and Y96. The Y96F variant, however, has a very close structural alignment.

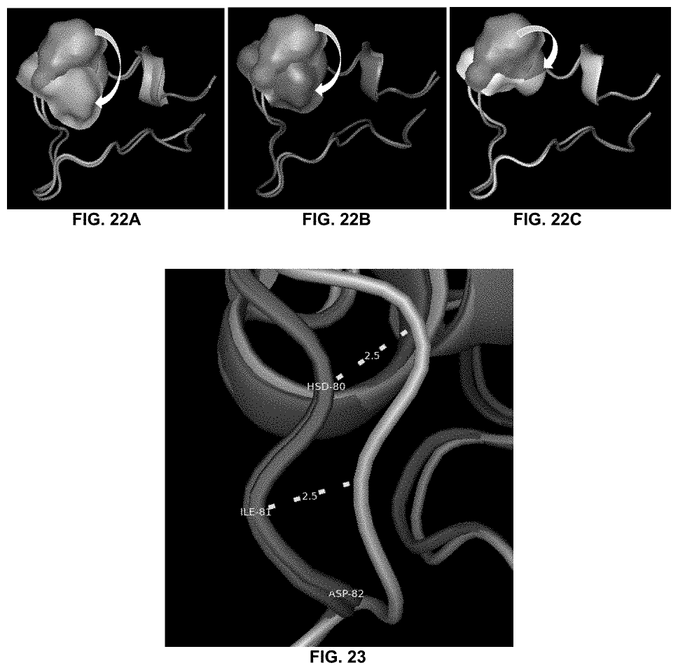

Each variant was then assessed to compare what the overlays revealed as compared to methyltransferase activity. This was initiated by viewing the overlays of G94D and K93E, both having complete zero methyltransferase activity, and used a space filling model to identify any residues that changed in both peptide simulations. It was discovered that the H80 residue in both G94D and K93E shifted clockwise approximately 90 degrees to an almost identical position in each variant (FIGS. 22A, 22B, and 22C).

Next, the other H80 residues in each variant were observed. In all cases except for the Y96“X” variants, the H80 residues displayed the same rotation of H80 into a similar position as the G94D and K93E variants. Finally, for the H80A variant, because it replaced the histidine with an alanine, the alanine positioning was explored. The alanine residue also moved but rotated approximately 45 degrees as opposed to the other H80 rotamers that were roughly 90 degrees pivoted. This H80 rotation was detected to also causes a knock-on effect that moves the, H80, I81, and D82 residues further to the left as compared to the wild type (FIG. 23). This effect was calculated to cause approximately a 2.5 Angstrom alteration in the variants R78A and R78G. As compared to histidine, alanine does not have a positive charge and it is hydrophobic, but it too caused the knock-on shift. Therefore, it was reasoned that because the variants that caused this shift had middle to low MTase values, any peptide containing this phenotype was ruled out as contenders for future testing as they would possibly affect methyltransferase activity. Any future variant containing the knock-on effect was to be dismissed as a potential therapeutic peptide. In the variants containing a Y96“X” change, Y96V, Y961, Y96A, T96F, the H80 residue did not shift. Consequently, other structural changes were sought out that may have negatively affected the methyltransferase activity in these variants.

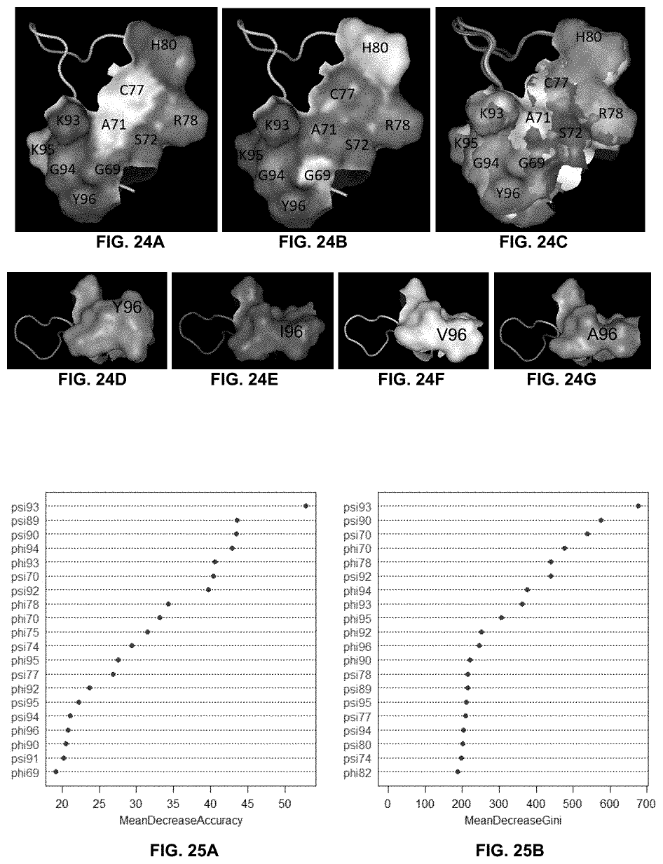

FIGS. 24A through 24G illustrate a comparison of the Y96“X” variants to the interaction regions of nsp10 to nsp14, and to nsp16. FIG. 24A illustrates WT peptide showing residues that interact with nsp14 in red. FIG. 24B illustrates WT residues that interact with nsp16 are in red. FIG. 24C illustrates Y96V white, Y961 magenta, Y96A orange, Y95F cyan, and WT green overlay showing the differences in the variants. Notable qualitative changes include the A71 residue protruding out further than the wild-type in all variants, including Y96F. For the Y96F variant that was the only notable change except for the F96 residue itself. FIGS. 24D through 24G illustrate other variants Y96V, Y961, and Y96A, where the A71 residue changed, protruding out further. Most notably is the change in Y96 to V, I or A, itself. In all cases the molecule loses a great deal of mass, and the protrusion that is seen in the wild-type contracts drastically. It was observed that all Y96“X” variants displayed an S72 residue that protrudes out further than wild-type, including Y96F. For the Y96F variant, that was the only notable change except for the F96 residue itself. In the other variants, Y96V, Y961, and Y96A, the A71 residue changed, also protruding out further. But again, the most notable change, is the change in Y96 to V, I or A, itself. In all cases, the R group of the molecule loses a great deal of mass, and the protrusion that is seen in the wild type does not contract as drastically. For Y96F, it should be noted that this variant is also found in many other coronavirus sequences and was the only variant with increased MTase activity.

Structurally, for the new variants, they are all mostly unremarkable. It was found that none had the H80 rotation, and none appeared to shift the 180 residue. For the Y96“X” variants, Y96C and Y96 W, again unremarkable, other than the Y96 residue. Y96C is quite a bit smaller than Y96, and Y96 W, as per the R group is larger than Y96.

As illustrated in the plots of FIGS. 25A and 25B, the random forest algorithm performed on the phi and psi dihedral angles revealed that the top 20 primary torsion predictors of accuracy for classification were residues located in the 70's and 90's portion of the peptide, with psi89 being the exception, situated adjacent to the highly conserved C90 residue. The C90 amino acid is also involved in zinc chelation and the psi90 angle is the third most accurate from the algorithm. Two other residues involved in zinc chelation were also in the top 15 angles for accuracy, psi74 and psi77. Phi 82, located next to the fourth chelating residue H83, was found on the Gini index. Thus, most of the amino acid residues are in the 90's and 70's, with, C74, C77, and C90 involved in the chelation of zinc.

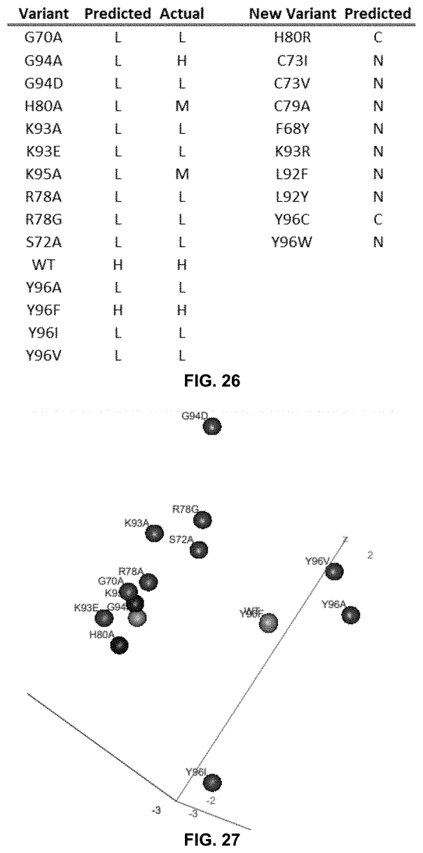

When using random forest for the prediction of MTase activity using the MDPPM, it predicted with 80% accuracy using the leaving one out strategy and designating three separate classes as “low,” “middle,” or “high,” according to MTase activity (FIG. 26). When using binomial “high” or “low” classification predictions, the sensitivity was 100%, the specificity 67%, and the accuracy 93%. In this case, the variant was classified as “low” if the variant decreases the MTase activity lower than 80%.

In the PCA analysis, on the variants with empirical data, the 3D components of the system and their distances from each other were visualized, as illustrated in FIG. 27. The WT, Y96F, and G94A (green) were designated as having “high” MTase activity, >80%. H80A and K95A were classified as “medium” MTase values (blue), i.e., those between 40-80%. The medium values clustered together and were at a generally closer distance to the high values cluster center, versus the “low” variants (red). Qualitatively, one can see that the Y96F and WT distances were very close.

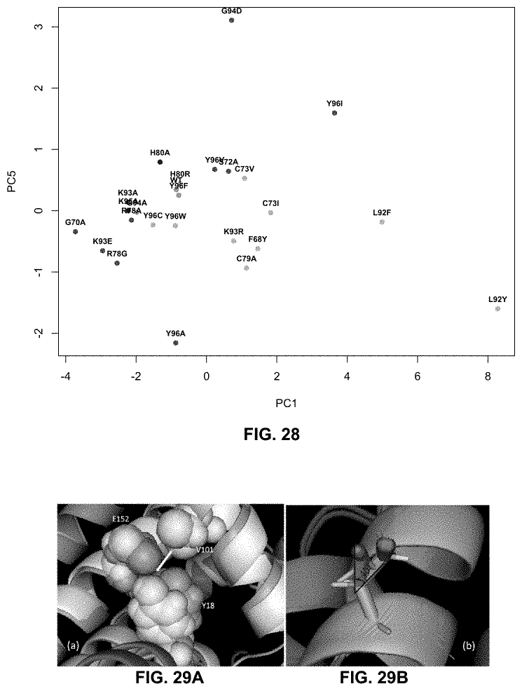

For the newly proposed variants, an additional PCA analysis was performed, one that included all variants, including new variants to be tested (cyan). FIG. 28 illustrates a 2D PCA plot for visual analysis. Variants with high MTase are in green, medium, blue, and those in red have low MTase activity. New variants tended to be clustered lowered and to the right. New variants closest to the green cluster, Y96C and H80 were predicted by random forest to be candidates for future testing. The random forest algorithm was used to find that PC1 and PC5 clustered the most accurately, and that the new variants were typically clustered away from the other variants in their own PC space.

5. Discussion

The emergence of new computational methods for drug development have begun to generate potential therapeutic compound structures at an unprecedented rate. One of the largest remaining challenges in peptide development is finding worthwhile new candidates amongst the sheer number of molecular possibilities. Currently, there are a few canonical approaches into how new drugs are designed and sifted through using computation methods and software. Molecular modeling, structure-based drug design, structure-based virtual screening, ligand-based modeling, and molecular dynamics are all used to help determine the relationship between the ligand and the target. Here, a multi-faceted approach was taken by Applicant by combining the molecular modeling method with structure-based drug design, while using molecular dynamics to assess the structure in solvent, all leading to machine learning data analytics for predictive measures.

Other computational studies relating to peptide development for Sars-Cov-2 have been performed, but they differ greatly in their scope and success. Can et al. only go so far as to say that nsp10 epitopes will be good candidates for vaccines. Sk et al. investigate the nsp10/nsp16 interface, but only recommend residues that might be investigated in the future such as G69, A71, G70, R78, Y96, three of those were used in this research. Others have done this as well, only venturing to propose hot spots. Ling and Sitthiyotha developed peptide models for Sars-Cov-2, but only were able to suggest new molecules based on molecular docking models. Furthermore, these models were not qualified or evaluated against wet lab data. Docking programs are known in the art to be notoriously inaccurate, with the best scoring at 50.0-60.0% accuracies for pose predictions, and with relatively weak correlations between docking scores and observed binding affinities. The advantage of the disclosure was that the peptide was already in the proper pose and that the binding energies in our model are predicted to be close to wild-type energies because they already exist in nature.

Two of the main challenges currently in the development of antiviral and antimicrobial drugs include the emergence of multidrug resistance and the transmission of drug-resistant strains from patient to patient that limit the clinical efficacy of current therapy. For emerging peptide drugs, one of the largest problems is the increased proteolytic instability as compared to not small molecules and monoclonal antibody therapeutics. Advantages to this model are that the stability can be accounted for in the simulations and that the epitope of the peptide is conserved. Further, once peptides are elucidated from the MDPPM, experimental testing using wet lab techniques such as the serum stability assay could be beneficial, future correlations to peptide stability to the MDPPM should be measured in future studies.

Current treatments of SARS-CoV-2 include two prominent drugs that were used for other indications, hydroxychloroquine (HCQ) and Remdesivir (RDV). HCQ has had mixed results in Covid19 treatment, however, when coupled with azithromycin resulted in a good clinical outcome and a virological cure in 973 out of 1061 patients within 10 days (91.7%). Notably, of those, nine developed QT prolongation but did not die of cardiotoxicity, rare cardiotoxicity adverse events are a large concern among the public. RDV in a compassionate use clinical study showed that there were clinical improvements in 67.9% of patients but that 60.3% had adverse events that included enzyme elevation and renal impairment. Both drugs demonstrate the need for alternative treatments due to safety measures and uncertain effectiveness, hence the need for nsp10/nsp16 conserved region interface drugs that target the virus machinery with higher safety and efficacy profiles.

Two candidates were identified that are almost 100 percent likely to have greater than 80% MTase activity, and, hopefully, greater than 100%. These variants deemed as candidates for further study had Euclidean distances that were very close to the “high” activity cluster. Applicant has very high confidence in these two candidates. Nevertheless, one cannot completely write off the other eight new variants as new candidate possibilities because MTase data for these have not yet been produced. In fact, it is recommended that should it be possible, these variants should be studied in a wet lab environment to gain insights into their potential to bind nsp16 or nsp14. This would further add to the accuracy and fine-tuning of the MDPPM. The new variants were found to have created their own cluster, which was further away from the deleterious variants. It is not beyond the realm of possibilities that the variant found furthest away from the “low” MTase variants, as well as the wild-type, L92Y, may be the variant that has the most potential to increase the affinity for nsp16. Nonetheless, one can only safely say, for now, that variants closest to WT and those predicted by our algorithm are the ones likely to have high MTase activity.

The findings of the phi and psi angles by random forest for accuracy discovered that they were in the 70's and 90's portion of the peptide, suggesting that these residues are important structurally and that changes in residues here may cause a decrease in MTase activity. It should be noted that residues in the 80's portion of the peptide are mostly semi-conserved, and that they are not as conserved as those in the 70's and 90's, which may also be a contributing factor to these results. Further, it was found that the chelating residues of C74, C77, and C90, located in the zinc finger, were in the top 20 phi and psi angles for accuracy predictions, and that D82, adjacent to the fourth chelating molecule H83, was in the Gini index, which calculates the data taking part in decision making. If the peptide cannot chelate to zinc it most likely loses function, therefore peptides affecting these residues or those adjacent may not be good picks for future peptide candidates.

Structurally and qualitatively, it was discerned that variants which had a rotamer at the H80 position had decreased MTase activity. This rotation also caused a knock-on effect which moved 181 and D82 away from the WT location. The H80 rotation is significant because even though H80 is not involved in binding nsp16, a rotation towards the C77 molecule may well affect the binding of nsp16, because C77 is directly involved in the nsp10/nsp16 interaction. And this may also be why nsp16 MTase activity is not as affected as nsp14 in the H80A variant, because it can only affect nsp16 indirectly through the C77 residue. Converse to nsp16, the H80 residue is directly involved in nsp14 binding, so any movement in this case is detrimental.

The Y96“X” residue variants' most important change seemed to be the Y96 amino acid itself. Although the other variants had a slight change qualitatively in the A71 residue, which is involved in the nsp16 interaction, there are two possibilities for the decrease in MTase activity, either subtle changes in the entire structure add up to an overall change that affects binding, or, that Y96 is important for a different reason, possibly biochemically. Furthering the mystery of the Y96 residue is that the size of the molecule is most likely not related to the adverse MTase effects one sees in the Y96“X” variants. This is because the Y96C cystine variant, also found in nature, was one of only two viable candidates that was obtained which had scored as high on the random forest predictions and had a close Euclidean distance to the WT.

6. Summary

Molecular dynamics coupled with machine learning can afford a way to predict whether new candidate peptides will have functional changes that affect methyltransferase activity. This study, as disclosed herein, has described a computer-implemented method in accordance with one or more embodiments to produce compelling new peptide leads computationally. Where there are distinct structural changes in variants, as with the H80 rotamer, one can visually see the differences that each variant creates and rule out any subsequent variants with the same phenotype. As is the case with Y96, however, when there are no distinct structural changes, one can rely on the specificity and accuracy of the computer-implemented method in accordance with one or more embodiments. It was shown that with the nsp10 peptide, precise residues were predicted that affect the binding of nsp16, and these need to be considered when developing new sequences.

The computer-implemented method in accordance with one or more embodiments shows promise in eliminating variants that may be of no scientific use, or of any use to those having limited resources who may only be able to test one or two sequences at a time. With the potential of septillions of residues in this small sequence alone, simulations coupled with machine learning can be of great use to scientists in the expedition of new drug leads.

Computer-Implemented Methods

Illustrated examples shown in FIGS. 42 to 46 set forth example computer-implemented methods 4200 through 5200. The respective flowcharts of the example computer-implemented methods 4200 through 5200 may be implemented by the one or more processors of one or more computing devices. In particular, the example computer-implemented methods 4200 through 5200 may be implemented as one or more modules in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof.

In the example computer-implemented methods 4200 through 5200, software executed by the one or more computing devices provides functionality described or illustrated herein. In particular, software executed by the one or more processors of the one or more computing devices is configured to perform one or more processing blocks of the example computer-implemented methods 4200 through 5200 set forth, described, and/or illustrated herein, or provides functionality set forth, described, and/or illustrated.

In the illustrated example embodiment of FIG. 42, illustrated process block 4202 includes selecting a reference wild-type non-mutated macromolecule.

The computer-implemented method 4200 may then proceed to illustrated process block 4204, which includes generating an ensemble of structures of the reference wild-type non-mutated macromolecule.

The computer-implemented method 4200 may then proceed to illustrated process block 4206, which includes providing an inhibitory constant for the phenotype structure of the reference wild-type non-mutated macromolecule.

The computer-implemented method 4200 may then proceed to illustrated process block 4208, which includes identifying, by executing one or more molecular dynamics simulations, one or more variant genomic structures of the reference wild-type macromolecule and acquiring measured inhibitory constants.

The computer-implemented method 4200 can terminate or end after execution of illustrated process block 4208.

Alternatively or additionally, the computer-implemented method 4200 may then proceed to illustrated process block 4210, which includes, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Alternatively or additionally, the computer-implemented method 4200 may then proceed to illustrated process block 4212, which includes, after identifying the one or more variant genomic structures, ranking functional effects of one or more variant macromolecules that correspond to the identified one or more variant genomic structures with respect to the corresponding wild-type macromolecule.

Alternatively or additionally, the computer-implemented method 4200 may then proceed to illustrated process block 4214, which includes training a machine learning model using the identified one or more variant genomic structures of the reference wild-type macromolecule and the measured inhibitory constants.

Alternatively or additionally, the computer-implemented method 4200 may then proceed to illustrated process block 4216, which includes classifying, via the trained machine learning model applied to the phi and psi dihedral angles of the amino acid BCL-2 protein, the identified one or more variant genomic structures.

Alternatively or additionally, the computer-implemented method 4200 may then proceed to illustrated process block 4218, which includes predicting via the trained machine learning model, an accuracy for resistance of each classified feature of the identified one or more variant genomic structures to a drug.

Alternatively or additionally, the computer-implemented method 4200 may then proceed to illustrated process block 4220, which includes predicting, via the trained machine learning model, a severity of drug resistance of each classified feature of the identified one or more variant genomic structures to a drug.

The computer-implemented method 4200 can terminate or end after execution of illustrated process block 4220.

In the illustrated example embodiment of FIG. 43, illustrated process block 4302 includes generating an ensemble of structures, e.g. the collection of structures that represents the range of conformation a protein assumes, of a reference wild-type non-mutated macromolecule.

The computer-implemented method 4300 may then proceed to illustrated process block 4304, which includes identifying one or more variant genomic structures of the reference wild-type macromolecule and acquiring measured inhibitory constants.

Alternatively or additionally, the computer-implemented method 4300 may then proceed to illustrated process block 4306, which includes, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Alternatively or additionally, the computer-implemented method 4300 may then proceed to illustrated process block 4308, which includes, after identifying the one or more variant genomic structures, ranking functional effects of one or more variant macromolecules that correspond to the identified one or more variant genomic structures with respect to the corresponding wild-type macromolecule

The computer-implemented method 4300 may then proceed to illustrated process block 4310, which includes training a machine learning model using the identified one or more variant genomic structures of the reference wild-type macromolecule and the measured inhibitory constants

The computer-implemented method 4300 may then proceed to illustrated process block 4312, which includes classifying, via the trained machine learning model applied to the phi and psi dihedral angles of the amino acid BCL-2 protein, the identified one or more variant genomic structures.

The computer-implemented method 4300 may then proceed to illustrated process block 4314, which includes predicting, via the trained machine learning model, an accuracy for drug resistance of each classified feature of the identified one or more variant genomic structures.

The computer-implemented method 4300 can terminate or end after execution of illustrated process block 4314.

In the illustrated example embodiment of FIG. 44, illustrated process block 4402 includes selecting a reference wild-type non-mutated macromolecule.

The computer-implemented method 4400 may then proceed to illustrated process block 4404, which includes generating an ensemble of structures of the reference wild-type non-mutated macromolecule.

The computer-implemented method 4400 may then proceed to illustrated process block 4406, which includes identifying a peptide having an antiviral effect or an antibacterial effect for use in treating infection from a bacterium or a virus.

In accordance with the computer-implemented method 4400, the identified peptide has an antiviral effect on SARS-CoV-2.

In accordance with the computer-implemented method 4400, identifying the peptide is performed by executing one or more molecular dynamics simulations.

In accordance with the computer-implemented method 4400, the one or more molecular dynamics simulations is executed using a Reference Protein Data Bank (PDB) Structure and Structural Variant Selection computational model.

In accordance with the computer-implemented method 4400, the one or more molecular dynamics simulations is executed using Nanoscale Molecular Dynamics (NAMD).

The computer-implemented method 4400 can terminate or end after execution of illustrated process block 4406.

In the illustrated example embodiment of FIG. 45, illustrated process block 4402 includes generating an ensemble of structures of a reference wild-type non-mutated macromolecule.

The computer-implemented method 4500 may then proceed to illustrated process block 4504, which includes identifying a peptide having an antiviral effect or an antibacterial effect for use in treating infection from a bacterium or a virus.

In accordance with the computer-implemented method 4500, the identified peptide has an antiviral effect on SARS-CoV-2.

In accordance with the computer-implemented method 4500, identifying the peptide is performed by executing one or more molecular dynamics simulations.

In accordance with the computer-implemented method 4500, the one or more molecular dynamics simulations is executed using a Reference Protein Data Bank (PDB) Structure and Structural Variant Selection computational model.

In accordance with the computer-implemented method 4500, the one or more molecular dynamics simulations is executed using Nanoscale Molecular Dynamics (NAMD).

The computer-implemented method 4500 can terminate or end after execution of illustrated process block 4504.

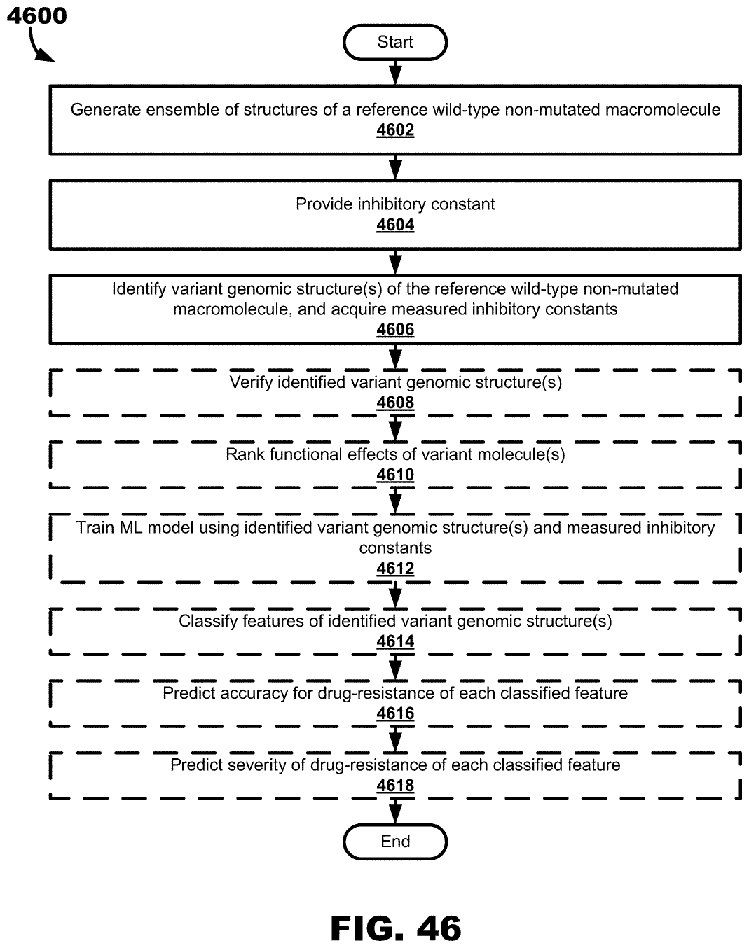

In the illustrated example embodiment of FIG. 46, illustrated process block 4602 includes generating an ensemble of structures of a reference wild-type non-mutated macromolecule.

The computer-implemented method 4600 may then proceed to illustrated process block 4604, which includes providing an inhibitory constant for the phenotype structure of the reference wild-type non-mutated macromolecule.

The computer-implemented method 4600 may then proceed to illustrated process block 4606, which includes identifying, by executing one or more molecular dynamics simulations, one or more variant genomic structures of the reference wild-type macromolecule and acquiring measured inhibitory constants.

In accordance with the computer-implemented method 4600, the one or more molecular dynamics simulations is executed using Nanoscale Molecular Dynamics (NAMD).

In accordance with the computer-implemented method 4600, the one or more variant macromolecules are variant proteins.

In accordance with the computer-implemented method 4600, the one or more variant proteins have been determined to cause a trait of a disease.

In accordance with the computer-implemented method 4600, the one or more variant proteins have been determined to cause a trait of a genetic disease.

In accordance with the computer-implemented method 4600, the disease is cancer.

In accordance with the computer-implemented method 4600, the macromolecule is BCL-2.

In accordance with the computer-implemented method 4600, the disease is Alzheimer's.

In accordance with the computer-implemented method 4600, the macromolecule is amyloid β peptide.

The computer-implemented method 4600 can terminate or end after execution of illustrated process block 4606.

Alternatively or additionally, the computer-implemented method 4600 may then proceed to illustrated process block 4608, which includes, after identifying the one or more variant genomic structures, verifying the one or more identified variant genomic structures using in-silica empirical data that is correlated and compared to X-ray crystallography for a structural analysis and surface plasmon resonance (SPR) for kinetic measurements of the inhibitory constant.

Alternatively or additionally, the computer-implemented method 4600 may then proceed to illustrated process block 4610, which includes, after identifying the one or more variant genomic structures, ranking functional effects of one or more variant macromolecules that correspond to the identified one or more variant genomic structures with respect to the corresponding wild-type macromolecule.

Alternatively or additionally, the computer-implemented method 4600 may then proceed to illustrated process block 4612, which includes training a machine learning model using the identified one or more variant genomic structures of the reference wild-type macromolecule and the measured inhibitory constants.

Alternatively or additionally, the computer-implemented method 4600 may then proceed to illustrated process block 4614, which includes classifying, via the trained machine learning model applied to the phi and psi dihedral angles of the amino acid BCL-2 protein, the features of the identified one or more variant genomic structures.

Alternatively or additionally, the computer-implemented method 4600 may then proceed to illustrated process block 4616, which includes predicting via the trained machine learning model, an accuracy for resistance of each classified feature of the identified one or more variant genomic structures to a drug.

Alternatively or additionally, the computer-implemented method 4600 may then proceed to illustrated process block 4618, predicting via the trained machine learning model, a severity of drug resistance of each classified feature of the identified one or more variant genomic structures to a drug.

In accordance with the computer-implemented method 4600, the drug comprises venetoclax.

The computer-implemented method 4600 can terminate or end after execution of illustrated process block 4618.

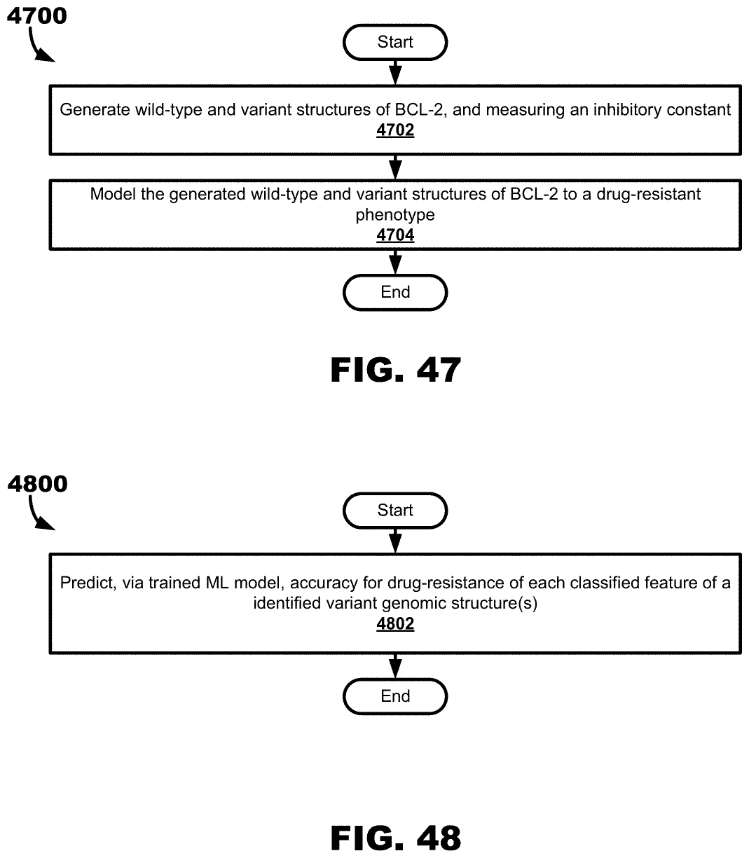

In the illustrated example embodiment of FIG. 47, illustrated process block 4702 includes generating, by executing one or more molecular dynamics simulations, one or more wild-type and variant genomic structures of BCL-2, and measuring an inhibitory constant using structural deviations to a binding cleft.

In accordance with the computer-implemented method 4700, the one or more molecular dynamics simulations is executed using Nanoscale Molecular Dynamics (NAMD).

In accordance with the computer-implemented method 4700, the inhibitory constant is Kᵢ.

In accordance with the computer-implemented method 4700, the inhibitory constant is K_{d}.

The computer-implemented method 4700 may then proceed to illustrated process block 4704, which includes modeling, by executing one or more molecular dynamics simulations, the generated one or more wild-type and variant genomic structures of BCL-2 to a drug-resistant phenotype.

In accordance with the computer-implemented method 4700, Replica Exchange Molecular Dynamics (REMD) simulations are used to model the one or more BCL-2 variant genomic structures and the wild-type reference macromolecule structure to the drug resistant phenotype.

The computer-implemented method 4700 can terminate or end after execution of illustrated process block 4704.

In the illustrated example embodiment of FIG. 48, illustrated process block 4802 includes predicting, via a trained machine learning model, an accuracy for drug resistance of each classified feature of an identified one or more variant genomic structures.

In accordance with the computer-implemented method 4800, each classified feature of the identified one or more variant genomic structures was classified via the trained machine learning model.

In accordance with the computer-implemented method 4800, the machine learning model was trained using the identified one or more variant genomic structures of a generated ensemble of genomic structures of a reference wild-type non-mutated macromolecule.

In accordance with the computer-implemented method 4800, the machine learning model was trained using measured inhibitory constants.

In accordance with the computer-implemented method 4800, the machine learning model was trained using measured inhibitory constants and the identified one or more variant genomic structures of a generated ensemble of genomic structures of a reference wild-type non-mutated macromolecule.

In accordance with the computer-implemented method 4800, the drug comprises Venetoclax.

The computer-implemented method 4800 can terminate or end after execution of illustrated process block 4802.

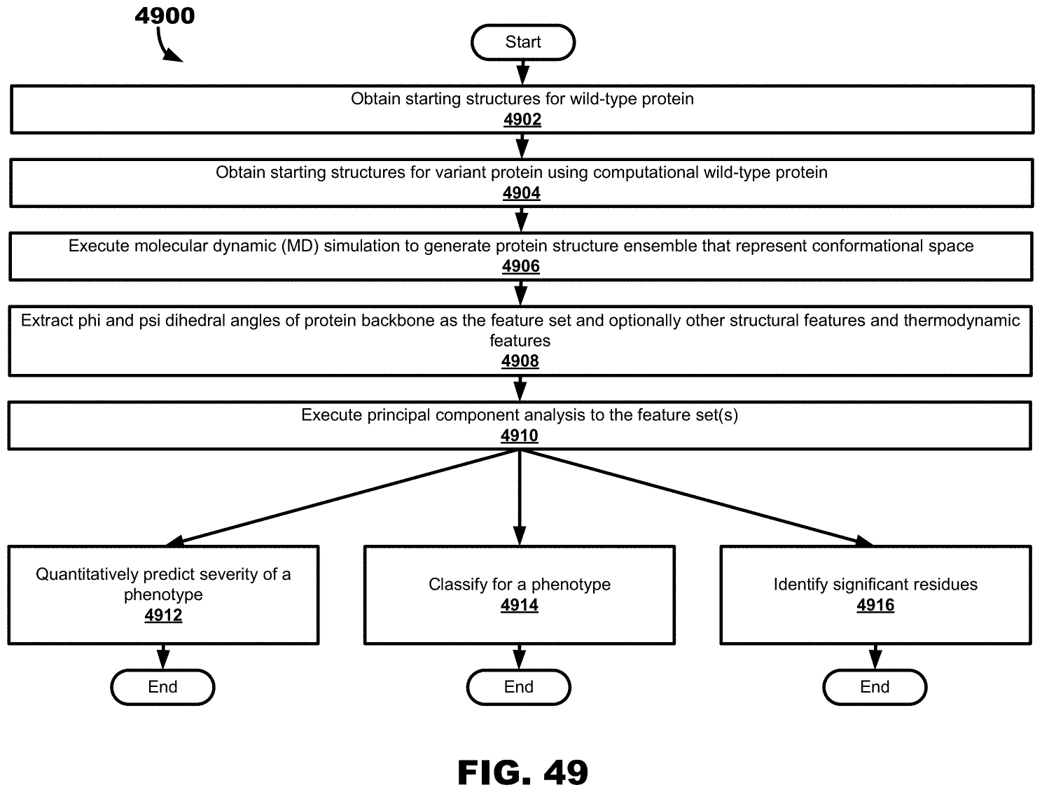

In the illustrated example embodiment of FIG. 49, illustrated process block 4902 includes obtaining one or more starting structures for a wild-type protein.

The computer-implemented method 4900 may then proceed to illustrated process block 4904, which includes obtaining one or more starting structures for a variant protein.

The computer-implemented method 4900 may then proceed to illustrated process block 4906, which includes generating an ensemble (collection) of protein structures that represent conformational space by executing a molecular simulation.

The computer-implemented method 4900 may then proceed to illustrated process block 4908, which includes obtaining a feature set by extracting from the phi and psi dihedral (torsional) angles of a protein backbone.

The computer-implemented method 4900 may then proceed to illustrated process block 4910, which includes executing a principal component analysis to the feature set.

After execution of process block 4910, the computer-implemented method 4900 may then proceed to illustrated process block 4912, which includes quantitatively predicting a severity of the phenotype using the derived equation. The computer-implemented method 4900 can terminate or end after execution of illustrated process block 4912.

Alternatively or additionally, after execution of process block 4910, the computer-implemented method 4900 may then proceed to illustrated process block 4914, which includes classifying, via the trained ML model, the variant protein with regard to phenotype. The computer-implemented method 4900 can terminate or end after execution of illustrated process block 4914.

Alternatively or additionally, after execution of process block 4910, the computer-implemented method 4900 may then proceed to illustrated process block 4916, which includes identifying, by applying one or more ML models, significant residues associated with pathology (i.e, which features are associated with a quantitative phenotype). The computer-implemented method 4900 can terminate or end after execution of illustrated process block 4916.

In accordance with the computer-implemented method 4900, the one or more starting structures for the wild-type protein are obtained from a protein data bank (PDB).

In accordance with the computer-implemented method 4900, the one or more starting structures for the wild-type protein are obtained via a computational prediction.

In accordance with the computer-implemented method 4900, the one or more starting structures for the variant protein are obtained using a computational prediction.

In accordance with the computer-implemented method 4900, the molecular simulation comprises an REMD simulation or an MD simulation.

In accordance with the computer-implemented method 4900, the feature set is obtained by extracting other structural and thermodynamic features.

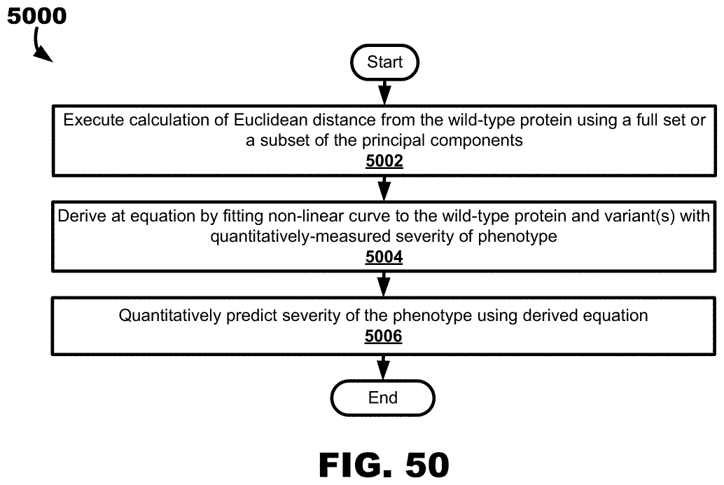

As illustrated in FIG. 50, the computer-implemented method 5000 is directed to quantitatively predicting a severity of the phenotype.

In the illustrated example embodiment of FIG. 50, illustrated process block 5002 includes executing a calculation of a Euclidean distance from the wild-type protein using a full set or a subset of the principal components.

The computer-implemented method 5000 may then proceed to illustrated process block 5004, which includes deriving at an equation by fitting a non-linear curve to the wild-type protein and the variant protein with a quantitatively-measured severity of phenotype.

The computer-implemented method 5000 may then proceed to illustrated process block 5006, which includes quantitatively predicting a severity of the phenotype using the derived equation.

The computer-implemented method 5000 can terminate or end after execution of illustrated process block 5006.

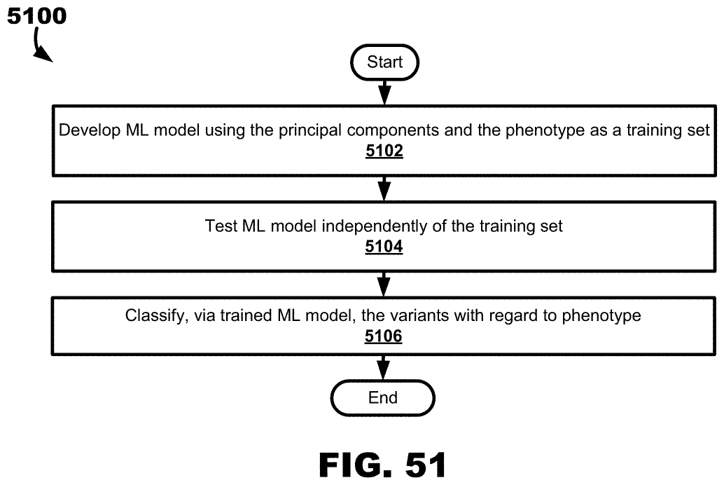

As illustrated in FIG. 51, the computer-implemented method 5100 is directed to classifying the genetic variants with regard to the phenotype.

In the illustrated example embodiment of FIG. 51, illustrated process block 5102 includes developing a machine learning (ML) model using the principal components and the phenotype as a training set.

The computer-implemented method 5100 may then proceed to illustrated process block 5104, which includes testing the ML model independently of the training set;

The computer-implemented method 5100 may then proceed to illustrated process block 5106, which includes classifying, via the trained ML model, the variant protein with regard to phenotype.

The computer-implemented method 5100 can terminate or end after execution of illustrated process block 5106.

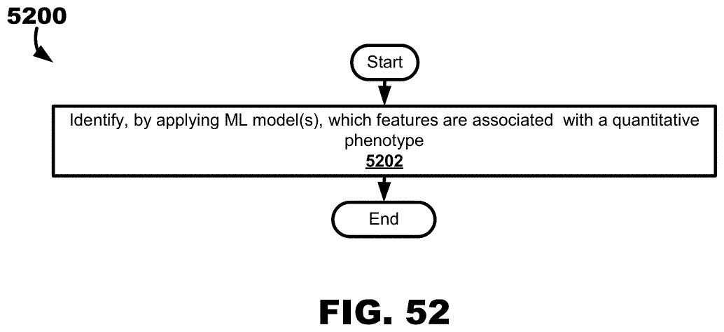

As illustrated in FIG. 52, the computer-implemented method 5200 is directed to identifying significant residues associated with a pathology.

In the illustrated example embodiment of FIG. 52, illustrated process block 5202 includes identifying, by applying one or more ML models, which features are associated with a quantitative phenotype.

The computer-implemented method 5200 can terminate or end after execution of illustrated process block 5202.

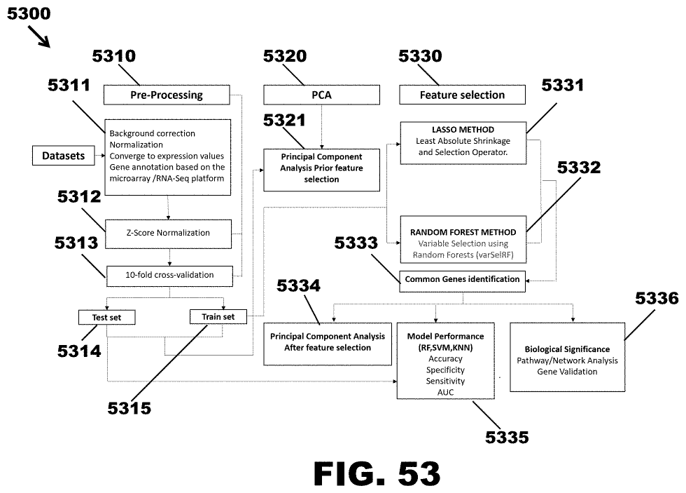

FIG. 53 illustrates a computer-implemented method that includes a pre-processing process block 5310, a principal component analysis (PCA) process block 5320, and a genetic feature selection process block 5330.

The pre-processing process block 5310 includes process block 5311, which includes processing of one or more data sets. Such data processing is to include, but is not limited to, background correction, normalization, convergence to expression values, and gene annotation based on a micro-array/RNA sequence platform.

After execution of process block 5311, the computer-implemented method 5300 may then proceed to illustrated process block 5312, which includes executing Z-score normalization of the data set.

After execution of process block 5312, the computer-implemented method 5300 may then proceed to illustrated process block 5313, which includes executing 10-fold cross validation of the data set.

After execution of process block 5312, the computer-implemented method 5300 may then proceed to illustrated process block 5314 and 5315, which includes testing and training the machine learning module using the data set.

The pre-processing process block 5320 includes process block 5321, which includes executing a principal component analysis (PCA) prior to feature selection, and process block 5322, which includes executing a principal component analysis (PCA) after feature selection.

The feature selection process block 5330 includes process block 5331, which includes executing Lasso method, and process block 5332, which includes executing the Forest tree method for the prediction of MTase activity using the MDPPM.

After execution of process blocks 5331 and 5332, the computer-implemented method 5300 may then proceed to illustrated process block 5333, which includes identifying common genes.

After execution of process blocks 5333, the computer-implemented method 5300 may then proceed to illustrated process block 5322 (PCA), process block 5322, which includes executing an evaluation of the training performance of the ML for diagnostic utility, and process block 5322, which includes identifying biologically significant residues associated with a pathology.

Clauses

Further, the disclosure comprises additional notes and examples as detailed in the following clauses:

Clause A

Clause 1A. A method, comprising: selecting a reference wild-type non-mutated macromolecule; generating an ensemble of genomic structures of the reference wild-type non-mutated macromolecule; providing an inhibitory constant for the phenotype structure of the reference wild-type non-mutated macromolecule; and identifying, by executing one or more molecular dynamics simulations, one or more variant genomic structures of the reference wild-type macromolecule, and acquiring measured inhibitory constants.

Clause 2A. The method of Clause 1A, further comprising, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Clause 3A, The method of Clause 1A, wherein the one or more molecular dynamics simulations is executed using Nanoscale Molecular Dynamics (NAMD).

Clause 4A. The method of Clause 1A, further comprising storing the identified one or more variant genomic structures of the reference wild-type macromolecule.

Clause 5A. The method of Clause 4A, further comprising training a machine learning model in response to storing the identified one or more variant genomic structures of the reference wild-type macromolecule.

Clause 6A. The method of Clause 5A, further comprising classifying, via the trained machine learning model applied to the phi and psi dihedral angles of the amino acid BCL-2 protein, the features of the identified one or more variant genomic structures.

Clause 7A. The method of Clause 6A, further comprising predicting via the trained machine learning model, an accuracy for resistance of each classified feature of the identified one or more variant genomic structures to a drug.

Clause 8A. The method of Clause 6A, further comprising predicting, via the trained machine learning model, a severity of drug resistance of each classified feature of the identified one or more variant genomic structures to a drug.

Clause 9A, The method of Clause 6A, wherein the drug comprises venetoclax.

Clause 10A. The method of Clause 1A, further comprising, after identifying the one or more variant genomic structures, ranking functional effects of one or more variant macromolecules that correspond to the identified one or more variant genomic structures with respect to the corresponding wild-type macromolecule.

Clause 11A. The method of Clause 1A, wherein the one or more variant macromolecules are variant proteins.

Clause 12A. The method of Clause 11A, wherein the one or more variant proteins have been determined to cause a trait of a disease.

Clause 13A. The method of Clause 12A, wherein the one or more variant proteins have been determined to cause a trait of a genetic disease.

Clause 14A. The method of Clause 12A, wherein the disease is cancer.

Clause 15A. The method of Clause 14A, wherein the macromolecule is BCL-2.

Clause 16A. The method of Clause 12A, wherein the disease is Alzheimer's.

Clause 17A. The method of Clause 16A, wherein the macromolecule is amyloid β peptide.

Clause 18A. The method of Clause 1A, further comprising: generating, by executing one or more molecular dynamics simulations, one or more wild-type and variant genomic structures of BCL-2 and measuring the inhibitory constant using structural deviations to a binding cleft.

Clause 19A. The method of Clause 18A, wherein the one or more molecular dynamics simulations is executed using Nanoscale Molecular Dynamics (NAMD).

Clause 20A. The method of Clause 19A, wherein the inhibitory constant is Kᵢ.

Clause 21A. The method of Clause 19A, wherein the inhibitory constant is K_{d}.

Clause 22A. The method of Clause 19A, further comprising modeling, by executing one or more molecular dynamics simulations, the one or more BCL-2 variant genomic structures and the wild-type reference macromolecule structure to a drug resistant phenotype.

Clause 23A. The method of Clause 22A, wherein the one or more molecular dynamics simulations is executed using Replica Exchange Molecular Dynamics (REMD).

Clause 24A. The method of Clause 19A, further comprising determining, by executing a General Born Implicit Solvent (GBIS) model, simulations of BCL-2 variant genomic variants that are executed by REMD.

Clause 25A. A computer-implemented method, comprising: generating an ensemble of genomic structures of a reference wild-type non-mutated macromolecule; identifying one or more variant genomic structures of the reference wild-type macromolecule, and acquiring measured inhibitory constants; storing the identified one or more variant genomic structures of the reference wild-type macromolecule; training a machine learning model in response to storing the identified one or more variant genomic structures of the reference wild-type macromolecule; classifying, via the trained machine learning model applied to the phi and psi dihedral angles of the amino acid BCL-2 protein, the features of the identified one or more variant genomic structures; and predicting, via the trained machine learning model, an accuracy for drug resistance of each classified feature of the identified one or more variant genomic structures.

Clause 26A. The computer-implemented method of Clause 25A, further comprising, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Clause 27A. The computer-implemented method of Clause 25A, wherein identifying the one or more variant genomic structures of the reference wild-type macromolecule is performed by executing one or more molecular dynamics simulations.

Clause 28A. The computer-implemented method of Clause 27A, wherein the one or more molecular dynamics simulations is executed using Nanoscale Molecular Dynamics (NAMD).

Clause 29A. The computer-implemented method of Clause 25A, wherein the drug comprises Venetoclax.

Clause 30A. The computer-implemented method of Clause 25A, further comprising, after identifying the one or more variant genomic structures: ranking functional effects of one or more variant macromolecules that correspond to the identified one or more variant genomic structures with respect to the corresponding wild-type macromolecule.

Clause 31A. The computer-implemented method of Clause 25A, wherein the one or more variant macromolecules are variant proteins.

Clause 32A. The computer-implemented method of Clause 31A, wherein the one or more variant proteins cause a trait of a disease.

Clause 33A. The computer-implemented method of Clause 32A, wherein the one or more variant proteins cause a trait of a genetic disease.

Clause 34A. The computer-implemented method of Clause 32A, wherein the disease is cancer.

Clause 35A. The computer-implemented method of Clause 34A, wherein the macromolecule is BCL-2.

Clause 36A. The computer-implemented method of Clause 32A, wherein the disease is Alzheimer's.

Clause 37A. The computer-implemented method of Clause 36A, wherein the macromolecule is amyloid β peptide.

Clause 38A. The computer-implemented method of Clause 25A, further comprising: generating, by executing one or more molecular dynamics simulations, one or more wild-type and variant genomic structures of BCL-2 and measuring the inhibitory constant using structural deviations to a binding cleft.

Clause 39A. The computer-implemented method of Clause 38A, wherein the one or more molecular dynamics simulations is executed using Nanoscale Molecular Dynamics (NAMD).

Clause 40A. The computer-implemented method of Clause 39A, wherein the inhibitory constant is Kᵢ.

Clause 41A. The computer-implemented method of Clause 39A, wherein the inhibitory constant is K_{d}.

Clause 42A. The computer-implemented method of Clause 39A, further comprising modeling, by executing one or more molecular dynamics simulations, the one or more BCL-2 variant genomic structures and the wild-type reference macromolecule structure to a drug resistant phenotype.

Clause 43A. The computer-implemented method of Clause 32A, wherein the one or more molecular dynamics simulations is executed using Replica Exchange Molecular Dynamics (REMD).

Clause 44A. The computer-implemented method of Clause 39A, further comprising determining, by executing a General Born Implicit Solvent (GBIS) model, simulations of BCL-2 variant genomic variants that are executed by REMD.

Clause 45A. A method, comprising: selecting a reference wild-type non-mutated macromolecule; generating an ensemble of genomic structures of the reference wild-type non-mutated macromolecule; and identifying a peptide having an antiviral effect or an antibacterial effect for use in treating infection from a bacteria or a virus.

Clause 46A. The method of Clause 45A, wherein the identified peptide has an antiviral effect on SARS-CoV-2.

Clause 47A. The method of Clause 45A, wherein identifying the peptide is performed by executing one or more molecular dynamics simulations.

Clause 48A. The method of Clause 45A, wherein the one or more molecular dynamics simulations is executed using a Reference Protein Data Bank (PDB) Structure and Structural Variant Selection computational model.

Clause 49A. The method of Clause 45A, wherein the one or more molecular dynamics simulations is executed using Nanoscale Molecular Dynamics (NAMD).

Clause 50A. A computer-implemented method, comprising: generating an ensemble of genomic structures of a reference wild-type non-mutated macromolecule; and identifying a peptide having an antiviral effect or an antibacterial effect for use in treating infection from a bacteria or a virus.

Clause 51A. The computer-implemented method of Clause 50A, wherein the virus is SARS-CoV-2.

Clause 52A. The computer-implemented method of Clause 50A, wherein identifying the peptide is performed by executing one or more molecular dynamics simulations.

Clause 53A. The computer-implemented method of Clause 50A, wherein the one or more molecular dynamics simulations is executed using a Reference Protein Data Bank (PDB) Structure and Structural Variant Selection computational model.

Clause 55A. The computer-implemented method of Clause 50A, wherein the one or more molecular dynamics simulations is executed using a computationally generated initial structure.

Clause 56A. The computer-implemented method of Clause 50A, wherein the one or more molecular dynamics simulations is executed using Nanoscale Molecular Dynamics (NAMD).

Clause B

Clause 1B. A method, comprising: generating an ensemble of genomic structures of a reference wild-type non-mutated macromolecule; providing an inhibitory constant for the phenotype structure of the reference wild-type non-mutated macromolecule; and identifying, by executing one or more molecular dynamics simulations, one or more variant genomic structures of the reference wild-type macromolecule, and acquiring measured inhibitory constants.

Clause 2B. The method of Clause 1B, further comprising, after identifying the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Clause 3B. The method of Clause 1B, wherein the one or more molecular dynamics simulations is executed using Nanoscale Molecular Dynamics (NAMD).

Clause 4B. The method of Clause 1B, further comprising storing the identified one or more variant genomic structures of the reference wild-type macromolecule.

Clause 5B. The method of Clause 4B, further comprising training a machine learning model in response to storing the identified one or more variant genomic structures of the reference wild-type macromolecule.

Clause 6B. The method of Clause 5B, further comprising classifying, via the trained machine learning model applied to the phi and psi dihedral angles of the amino acid BCL-2 protein, the features of the identified one or more variant genomic structures.

Clause 7B. The method of Clause 6B, further comprising predicting via the trained machine learning model, an accuracy for resistance of each classified feature of the identified one or more variant genomic structures to a drug.

Clause 8B. The method of Clause 6B, further comprising predicting, via the trained machine learning model, a severity of drug resistance of each classified feature of the identified one or more variant genomic structures to a drug.

Clause 9B, The method of Clause 6B, wherein the drug comprises Venetoclax.

Clause 10B. The method of Clause 1B, further comprising, after identifying the one or more variant genomic structures, ranking functional effects of one or more variant macromolecules that correspond to the identified one or more variant genomic structures with respect to the corresponding wild-type macromolecule.

Clause 11B. The method of Clause 1B, wherein the one or more variant macromolecules are variant proteins.

Clause 12B. The method of Clause 11B, wherein the one or more variant proteins have been determined to cause a trait of a disease.

Clause 13B. The method of Clause 12, wherein the one or more variant proteins have been determined to cause a trait of a genetic disease.

Clause 14B. The method of Clause 12B, wherein the disease is cancer.

Clause 15B. The method of Clause 14B, wherein the macromolecule is BCL-2.

Clause 16B. The method of Clause 12B, wherein the disease is Alzheimer's.

Clause 17B. The method of Clause 16B, wherein the macromolecule is amyloid β peptide.

Clause 18B, The method of Clause 1B, further comprising: generating, by executing one or more molecular dynamics simulations, one or more wild-type and variant genomic structures of BCL-2; and measuring the inhibitory constant using structural deviations to a binding cleft.

Clause 19B. The method of Clause 18B, wherein the one or more molecular dynamics simulations is executed using Nanoscale Molecular Dynamics (NAMD).

Clause 20B. The method of Clause 19B, wherein the inhibitory constant is Ki.

Clause 21B. The method of Clause 19B, wherein the inhibitory constant is Kd.

Clause 22B. The method of Clause 19B, further comprising modeling, by executing one or more molecular dynamics simulations, the one or more BCL-2 variant genomic structures and the wild-type reference macromolecule structure to a drug resistant phenotype.

Clause 23B. The method of Clause 22B, wherein the one or more molecular dynamics simulations is executed using Replica Exchange Molecular Dynamics (REMD).

Clause 24B. The method of Clause 19B, further comprising determining, by executing a General Born Implicit Solvent (GBIS) model, simulations of BCL-2 variant genomic variants that are executed by REMD.

Clause 25B. A computer-implemented method, comprising: generating an ensemble of genomic structures of a reference wild-type non-mutated macromolecule; identifying one or more variant genomic structures of the reference wild-type macromolecule, and acquiring measured inhibitory constants; storing the identified one or more variant genomic structures of the reference wild-type macromolecule; training a machine learning model in response to storing the identified one or more variant genomic structures of the reference wild-type macromolecule; classifying, via the trained machine learning model applied to the phi and psi dihedral angles of the amino acid BCL-2 protein, the features of the identified one or more variant genomic structures; and predicting, via the trained machine learning model, a severity of drug resistance of each classified feature of the identified one or more variant genomic structures to a drug.

Clause 26B. The computer-implemented method of Clause 25B, further comprising, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Clause 27B. The computer-implemented method of Clause 25B, wherein identifying the one or more variant genomic structures of the reference wild-type macromolecule is performed by executing one or more molecular dynamics simulations.

Clause 28B. The computer-implemented method of Clause 27B, wherein the one or more molecular dynamics simulations is executed using Nanoscale Molecular Dynamics (NAMD).

Clause 29B. The computer-implemented method of Clause 25B, wherein the drug comprises Venetoclax.

Clause 30B. The computer-implemented method of Clause 25B, further comprising, after identifying the one or more variant genomic structures: ranking functional effects of one or more variant macromolecules that correspond to the identified one or more variant genomic structures with respect to the corresponding wild-type macromolecule.

Clause 31B. The computer-implemented method of Clause 25B, wherein the one or more variant macromolecules are variant proteins.

Clause 32B. The computer-implemented method of Clause 31B, wherein the one or more variant proteins cause a trait of a disease.

Clause 33B. The computer-implemented method of Clause 32B, wherein the one or more variant proteins cause a trait of a genetic disease.

Clause 34B. The computer-implemented method of Clause 32B, wherein the disease is cancer.

Clause 35B. The computer-implemented method of Clause 34B, wherein the macromolecule is BCL-2.

Clause 36B. The computer-implemented method of Clause 32B, wherein the disease is Alzheimer's.

Clause 37B. The computer-implemented method of Clause 36B, wherein the macromolecule is amyloid β peptide.

Clause 38B. The computer-implemented method of Clause 32B, wherein the one or more molecular dynamics simulations is executed using Replica Exchange Molecular Dynamics (REMD).

Clause 39B. A computer-implemented method, comprising: generating, by executing one or more molecular dynamics simulations, one or more wild-type and variant genomic structures of BCL-2; and measuring the inhibitory constant using structural deviations to a binding cleft.

Clause 40B. The computer-implemented method of Clause 39B, wherein the one or more molecular dynamics simulations is executed using Nanoscale Molecular Dynamics (NAMD).

Clause 41B. The computer-implemented method of Clause 40B, wherein the inhibitory constant is Ki.

Clause 42B. The computer-implemented method of Clause 40B, wherein the inhibitory constant is Kd.

Clause 43B. The computer-implemented method of Clause 40B, further comprising modeling, by executing one or more molecular dynamics simulations, the one or more BCL-2 variant genomic structures and the wild-type reference macromolecule structure to a drug resistant phenotype.

Clause 44B. The computer-implemented method of Clause 40B, further comprising determining, by executing a General Born Implicit Solvent (GBIS) model, simulations of BCL-2 variant genomic variants that are executed by REMD.

Clause C

Clause 25C. A computer-implemented method, comprising predicting, via a trained machine learning model, an accuracy for drug resistance of each classified feature of an identified one or more variant genomic structures, wherein each classified feature of the identified one or more variant genomic structures was classified via the trained machine learning model; and the machine learning model was trained with: the identified one or more variant genomic structures of a generated ensemble of genomic structures of a reference wild-type non-mutated macromolecule; and measured inhibitory constants.

Clause 26C. The computer-implemented method of Clause 25C, further comprising, after identifying the one or more variant genomic structures and before classifying the features of the one or more variant genomic structures: verifying the one or more identified variant genomic structures using in-silica empirical data that is correlated and compared to X-ray crystallography for a structural analysis and surface plasmon resonance (SPR) for kinetic measurements of the inhibitory constant.

Clause 27C. The computer-implemented method of Clause 25C, wherein identifying the one or more variant genomic structures of the reference wild-type macromolecule is performed by executing one or more molecular dynamics simulations.

Clause 28C. The computer-implemented method of Clause 27C, wherein the one or more molecular dynamics simulations is executed using Nanoscale Molecular Dynamics (NAMD).

Clause 29C. The computer-implemented method of Clause 25C, wherein the drug comprises Venetoclax.

Clause 30C. The computer-implemented method of Clause 25C, further comprising, after identifying the one or more variant genomic structures: ranking functional effects of one or more variant macromolecules that correspond to the identified one or more variant genomic structures with respect to the corresponding wild-type macromolecule.

Clause 31C. The computer-implemented method of Clause 25C, wherein the one or more variant macromolecules are variant proteins.

Clause 32C. The computer-implemented method of Clause 31C, wherein the one or more variant proteins cause a trait of a disease.

Clause 33C. The computer-implemented method of Clause 32C, wherein the one or more variant proteins cause a trait of a genetic disease.

Clause 34C. The computer-implemented method of Clause 32C, wherein the disease is cancer.

Clause 35C. The computer-implemented method of Clause 34C, wherein the macromolecule is BCL-2.

Clause 36C. The computer-implemented method of Clause 32C, wherein the disease is Alzheimer's.

Clause 37C. The computer-implemented method of Clause 36C, wherein the macromolecule is amyloid β peptide.

Clause 38C. The computer-implemented method of Clause 25C, further comprising: generating, by executing one or more molecular dynamics simulations, one or more wild-type and variant genomic structures of BCL-2; and measuring the inhibitory constant using structural deviations to a binding cleft.

Clause 39C. The computer-implemented method of Clause 38C, wherein the one or more molecular dynamics simulations is executed using Nanoscale Molecular Dynamics (NAMD).

Clause 40C. The computer-implemented method of Clause 39C, wherein the inhibitory constant is Kᵢ.

Clause 41C. The computer-implemented method of Clause 39C, wherein the inhibitory constant is K_{d}.

Clause 42C. The computer-implemented method of Clause 39C, further comprising modeling, by executing one or more molecular dynamics simulations, the one or more BCL-2 variant genomic structures and the wild-type reference macromolecule structure to a drug resistant phenotype.

Clause 43C. The computer-implemented method of Clause 32C, wherein the one or more molecular dynamics simulations is executed using Replica Exchange Molecular Dynamics (REMD).

Clause 44C. The computer-implemented method of Clause 39C, further comprising determining, by executing a General Born Implicit Solvent (GBIS) model, simulations of BCL-2 variant genomic variants that are executed by REMD.

Clause D

Clause 1 D. A computer-implemented method, comprising: obtaining one or more starting structures for a wild-type protein; obtaining one or more starting structures for a variant protein; generating an ensemble (collection) of protein structures that represent conformational space by executing a molecular simulation; obtaining a feature set by extracting from the phi and psi dihedral (torsional) angles of a protein backbone; and executing a principal component analysis to the feature set.

Clause 2D. The computer-implemented method of Clause 1D, wherein the one or more starting structures for the wild-type protein are obtained from a protein data bank (PDB).

Clause 3D. The computer-implemented method of Clause 1D, wherein the one or more starting structures for the wild-type protein are obtained via a computational prediction.

Clause 4D. The computer-implemented method of Clause 1D, wherein the one or more starting structures for the variant protein are obtained using a computational prediction.

Clause 5D. The computer-implemented method of Clause 1D, wherein the molecular simulation comprises an REMD simulation or an MD simulation.

Clause 6D. The computer-implemented method of Clause 1 D, wherein the feature set is obtained by extracting other structural and thermodynamic features.

Clause 7D. A computer-implemented method, comprising: obtaining one or more starting structures for a wild-type protein; obtaining one or more starting structures for a variant protein; generating an ensemble (collection) of protein structures that represent conformational space by executing a molecular simulation; obtaining a feature set by extracting from the phi and psi dihedral (torsional) angles of a protein backbone; executing a principal component analysis to the feature set; execute calculation of a Euclidean distance from the wild-type protein using a full set or a subset of the principal components; deriving at an equation by fitting a non-linear curve to the wild-type protein and the variant protein with a quantitatively-measured severity of phenotype; and quantitatively predicting a severity of the phenotype using the derived equation.

Clause 8D. The computer-implemented method of Clause 7D, wherein the one or more starting structures for the wild-type protein are obtained from a protein data bank (PDB).

Clause 9D. The computer-implemented method of Clause 7D, wherein the one or more starting structures for the wild-type protein are obtained via a computational prediction.

Clause 10D. The computer-implemented method of Clause 7D, wherein the one or more starting structures for the variant protein are obtained using a computational prediction.

Clause 11D. The computer-implemented method of Clause 7D, wherein the molecular simulation comprises an REMD simulation or an MD simulation.

Clause 12D. The computer-implemented method of Clause 7D, wherein the feature set is obtained by extracting other structural and thermodynamic features.

Clause 13D. A computer-implemented method, comprising: obtaining one or more starting structures for a wild-type protein; obtaining one or more starting structures for a variant protein; generating an ensemble (collection) of protein structures that represent conformational space by executing a molecular simulation; obtaining a feature set by extracting from the phi and psi dihedral (torsional) angles of a protein backbone; executing a principal component analysis to the feature set; developing a machine learning (ML) model using the principal components and the phenotype as a training set; testing the ML model independently of the training set; and classifying, via the trained ML model, the variant protein with regard to phenotype.

Clause 14D. The computer-implemented method of Clause 13D, wherein the one or more starting structures for the wild-type protein are obtained from a protein data bank (PDB).

Clause 15D. The computer-implemented method of Clause 13D, wherein the one or more starting structures for the wild-type protein are obtained via a computational prediction.

Clause 16D. The computer-implemented method of Clause 13D, wherein the one or more starting structures for the variant protein are obtained using a computational prediction.

Clause 17D. The computer-implemented method of Clause 13D, wherein the molecular simulation comprises an REMD simulation or an MD simulation.

Clause 18D. The computer-implemented method of Clause 13D, wherein the feature set is obtained by extracting other structural and thermodynamic features.

Clause 19D. A computer-implemented method, comprising: obtaining one or more starting structures for a wild-type protein; obtaining one or more starting structures for a variant protein; generating an ensemble (collection) of protein structures that represent conformational space by executing a molecular simulation; obtaining a feature set by extracting from the phi and psi dihedral (torsional) angles of a protein backbone; executing a principal component analysis to the feature set; and identifying, by applying one or more ML models, which features are associated with a quantitative phenotype.

Clause 20D. The computer-implemented method of Clause 19D, wherein the one or more starting structures for the wild-type protein are obtained from a protein data bank (PDB).

Clause 21D. The computer-implemented method of Clause 19D, wherein the one or more starting structures for the wild-type protein are obtained via a computational prediction.

Clause 22D. The computer-implemented method of Clause 19D, wherein the one or more starting structures for the variant protein are obtained using a computational prediction.

Clause 23D. The computer-implemented method of Clause 19D, wherein the molecular simulation comprises an REMD simulation or an MD simulation.

Clause 24D. The computer-implemented method of Clause 19D, wherein the feature set is obtained by extracting other structural and thermodynamic features.

The terms “coupled,” “attached,” or “connected” may be used herein to refer to any type of relationship, direct or indirect, between the components in question, and may apply to electrical, mechanical, fluid, optical, electromagnetic, electromechanical or other connections. Additionally, the terms “first,” “second,” etc. are used herein only to facilitate discussion, and carry no particular temporal or chronological significance unless otherwise indicated. The terms “cause” or “causing” means to make, force, compel, direct, command, instruct, and/or enable an event or action to occur or at least be in a state where such event or action may occur, either in a direct or indirect manner.

Those skilled in the art will appreciate from the foregoing description that the broad techniques of the exemplary embodiments may be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B, or C” means “any of the following: A; B; C; A and B; A and C; B and C; A, B, and C.” An exception to this definition will occur only when a combination of elements, functions, steps, or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

The use of any and all examples or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

What is claimed is:

1. A method, comprising:
generating an ensemble of genomic structures of a reference wild-type non-mutated macromolecule;
providing an inhibitory constant for the phenotype structure of the reference wild-type non-mutated macromolecule; and
identifying, by executing one or more molecular dynamics simulations, one or more variant genomic structures of the reference wild-type macromolecule and acquiring measured inhibitory constants.

2. The method of claim 1, further comprising training a machine learning model using the measured inhibitory constants and the identified one or more variant genomic structures of the reference wild-type macromolecule.

3. The method of claim 2, further comprising classifying, by applying the phi and psi dihedral angles of an amino acid BCL-2 protein to the trained machine learning model, the features of the identified one or more variant genomic structures.

4. The method of claim 3, further comprising predicting via the trained machine learning model, an accuracy for resistance of each classified feature of the identified one or more variant genomic structures to a drug.

5. The method of claim 4, further comprising predicting, via the trained machine learning model, a severity of resistance of each classified feature of the identified one or more variant genomic structures to the drug.

6. The method of claim 6, wherein the drug comprises Venetoclax.

7. The method of claim 1, further comprising, after identifying the one or more variant genomic structures, ranking functional effects of one or more variant macromolecules that correspond to the identified one or more variant genomic structures with respect to a corresponding wild-type macromolecule.

8. The method of claim 7, wherein the one or more variant macromolecules are variant proteins that have been determined to cause a trait of a disease.

9. The method of claim 8, wherein the disease is cancer.

10. The method of claim 9, wherein the one or more variant macromolecules is BCL-2.

11. The method of claim 8, wherein the disease is Alzheimer's.

12. The method of claim 11, wherein the one or more variant macromolecules is amyloid β peptide.

13. A computer-implemented method, comprising:
generating an ensemble of genomic structures of a reference wild-type non-mutated macromolecule;
identifying one or more variant genomic structures of the reference wild-type macromolecule, and acquiring measured inhibitory constants;
training a machine learning model using the identified one or more variant genomic structures of the reference wild-type macromolecule and the measured inhibitory constants;
classifying, via the trained machine learning model applied to the phi and psi dihedral angles of an amino acid BCL-2 protein, the features of the identified one or more variant genomic structures; and
predicting, via the trained machine learning model, an accuracy for resistance of each classified feature of the identified one or more variant genomic structures to a drug.

14. The method of claim 13, further comprising predicting, via the trained machine learning model, a severity of resistance of each classified feature of the identified one or more variant genomic structures to the drug.

15. The method of claim 14, wherein the drug comprises venetoclax.

16. The method of claim 13, further comprising, after identifying the one or more variant genomic structures, ranking functional effects of one or more variant macromolecules that correspond to the identified one or more variant genomic structures with respect to a corresponding wild-type macromolecule.

17. The method of claim 16, wherein the one or more variant macromolecules are variant proteins that have been determined to cause a trait of a disease.

18. The method of claim 17, wherein:
the disease is cancer, and
the one or more variant macromolecules is BCL-2.

19. The method of claim 17, wherein:
the disease is Alzheimer's, and
the one or more variant macromolecules is amyloid β peptide.

20. A computer-implemented method, comprising:
predicting, via a trained machine learning model, an accuracy for drug resistance of each classified feature of an identified one or more variant genomic structures,
wherein:
each classified feature of the identified one or more variant genomic structures was classified via the trained machine learning model, and
the machine learning model was trained with the identified one or more variant genomic structures of a generated ensemble of genomic structures of a reference wild-type non-mutated macromolecule and measured inhibitory constants.
